(19) United States
(12) Patent Application Publication
(10) Pub. No.: US 2013/0243883 A1
(43) Pub. Date: Sep. 19, 2013

(54) STABLE FORMULATIONS

(71) Applicant: REOXCYN DISCOVERIES GROUP, INC., Salt Lake City, UT (US)

(72) Inventors: Verdis Norton, Sandy, UT (US); Gary L. Samuelson, Sandy, UT (US)

(73) Assignee: Reoxcyn Discoveries Group, Inc., Salt Lake City, UT (US)

(21) Appl. No.: 13/857,882

(22) Filed: Apr. 5, 2013

Related U.S. Application Data
(63) Continuation-in-part of application No. 12/592,402, filed on Nov. 24, 2009, now Pat. No. 8,455,010.
Application No. 12/592,402 is a continuation-in-part of application No. 12/383,212, filed on Mar. 20, 2009, now Pat. No. 8,367,120.
Application No. 12/383,212 is a continuation-in-part of application No. 12/290,398, filed on Oct. 30, 2008, now abandoned.
Application No. 12/290,398 is a continuation-in-part of application No. 12/381,399, filed on Mar. 11, 2009, now abandoned.
Application No. 12/381,399 is a continuation-in-part of application No. 12/290,398, filed on Oct. 30, 2008, now abandoned.
(60) Provisional application No. 61/001,010, filed on Oct. 30, 2007.
(60) Provisional application No. 61/068,990, filed on Mar. 11, 2008.
(60) Provisional application No. 61/001,010, filed on Oct. 30, 2007.
(60) Provisional application No. 61/707,141, filed on Sep. 28, 2012.
(60) Provisional application No. 61/706,670, filed on Sep. 27, 2012.
(60) Provisional application No. 61/704,401, filed on Sep. 21, 2012.

(51) Int. Cl. A61K 33/40; C25B 1/04

(52) U.S. Cl. CPC A61K 33/40; C25B 1/04
USPC 424/615; 205/633

(57) ABSTRACT

Described herein are aqueous formulations with stabilized reactive and/or radical species.

### CROSS REFERENCE TO RELATED APPLICATIONS

This application is a continuation-in-part of U.S. patent application Ser. No. 12/592,402 filed Nov. 24, 2009, which is a continuation-in-part of U.S. patent application Ser. No. 12/383,212 filed Mar. 20, 2009 now U.S. Pat. No. 8,367,120, which is a continuation-in-part of U.S. patent application Ser. No. 12/290,398 filed Oct. 30, 2008, which claims the benefit of U.S. provisional patent application No. 61/001,010 filed Oct. 30, 2007, the disclosures each of which are incorporated herein by reference in their entirety.

This application is a continuation-in-part of U.S. patent application Ser. No. 12/381,399 filed Mar. 11, 2009 which claims the benefit of U.S. provisional patent application No. 61/068,990 filed Mar. 11, 2008 and which is a continuation-in-part of U.S. patent application Ser. No. 12/290,398 filed Oct. 30, 2008, which claims the benefit of U.S. provisional patent application No. 61/001,010 filed Oct. 30, 2007, the disclosures each of which are incorporated herein by reference in their entirety.

This application claims the benefit of U.S. provisional patent application Nos. 61/704,401 filed Sep. 21, 2012, 61/706,670 filed Sep. 27, 2012, and 61/707,141 filed Sep. 28, 2012, the disclosures each of which are incorporated herein by reference in their entirety.

### FIELD

The present invention relates to aqueous formulations including stable reactive species.

### SUMMARY

Described herein generally are aqueous formulations including at least one stable reactive and/or radical species. The aqueous formulations can be non-irritating when administered, non-toxic, or both. The aqueous formulations can comprise an electrolyzed saline solution housed in a non-reactive container, wherein the electrolyzed saline solution includes at least one radical that is greater than about 60% stable for at least 1 year. In some embodiments, the non-reactive container is a borosilicate glass bottle.

In some embodiments, the at least one radical can be any radical that would otherwise be non-stable. Such radical can include oxygen radicals such as but not limited to superoxide. For example, superoxide housed in a non-reactive borosilicate glass bottle can have a half-life of about 24 years.

The aqueous formulations can include the electrolyzed saline solution comprising superoxide and at least one hypochlorite. The aqueous formulations can be electrolyzed at a temperature between about 4.5° C. and about 5.8° C.

The aqueous formulations described can have unique components, such as stable superoxides, hydroxyl radicals and OOH*.

Methods of making aqueous formulations are also described comprising: electrolyzing a circulating salt solution using a set of electrodes thereby forming an electrolyzed saline solution; and placing the electrolyzed saline solution into a non-reactive bottle or container. In some embodiments, the electrolyzed saline solution can include at least one oxygen radical that is greater than about 60% stable for at least 1 year within the non-reactive container.

In one embodiment, methods are described of making an aqueous formulation comprising: electrolyzing a circulating, chilled solution of at least 1,000 gallons having a salt concentration of about 10.75 g NaCl/gal using a set of at least 7 electrodes with a total amperage of about 56 amps thereby forming an electrolyzed saline solution; and placing the electrolyzed saline solution into a non-reactive borosilicate glass bottle, wherein the electrolyzed saline solution includes superoxide that is greater than about 95% stable for at least 1 year within the non-reactive borosilicate glass bottle and at least one hypochlorite, wherein the electrolyzed saline solution is non-toxic, and wherein the electrolyzed saline solution is non-irritating.

### BRIEF DESCRIPTION OF THE DRAWINGS

FIG. 1 is a flow chart of a process as described herein.

FIG. 2 illustrates an example diagram of the generation of various molecules at the electrodes. The molecules written between the electrodes depict the initial reactants and those on the outside of the electrodes depict the molecules/ions produced at the electrodes and their electrode potentials.

FIG. 3A illustrates a plan view of a process and system for producing an aqueous formulation according to the present description. FIG. 3B illustrates a plan view of an example power supply.

FIG. 4 illustrates an example system for preparing water for further processing the aqueous formulation.

FIG. 5 illustrates a ³⁵Cl spectrum of NaCl, NaClO solution at a pH of 12.48, and the aqueous formulation.

FIG. 6 illustrates a ¹H NMR spectrum of an aqueous formulation as described.

FIG. 7 illustrates a ³¹P NMR spectrum of DIPPMPO combined with the beverage.

FIG. 8 illustrates a mass spectrum showing a parent peak and fragmentation pattern for DIPPMPO with m/z peaks at 264, 222, and 180.

FIG. 9 illustrates oxygen/nitrogen ratios for an aqueous formulation described herein compared to water and NaClO.

FIG. 10 illustrates chlorine/nitrogen ratios for an aqueous formulation described herein compared to water and NaClO.

FIG. 11 illustrates ozone/nitrogen ratios for an aqueous formulation described herein compared to water and NaClO.

FIG. 12 illustrates the carbon dioxide to nitrogen ratio of an aqueous formulation as described herein compared to water and NaClO.

FIG. 13 illustrates an EPR splitting pattern for a free electron.

FIG. 14 illustrates a graph of AAPH concentration versus area under the curve of the fluorescence signal.

FIG. 15 illustrates a graph of percent of remaining fluorescence versus time using AAPH.

FIG. 16 is a graph of bottle and pouch decay over time.

### DETAILED DESCRIPTION

Described herein are aqueous formulations, solutions, elixirs, and beverages. The aqueous formulations include an electrolyzed saline solution which can be housed in a non-reactive container or bottle. The electrolyzed saline solutions can include at least one stable radical, for example, an oxygen radical.

Generally, the aqueous formulations can be non-irritating when administered to a mammal, such as a human. Further, the aqueous solutions can be non-toxic when administered to a mammal, such as a human. In some embodiments, non-irritating and non toxic can be in reference to recommended administration amounts.

The electrolyzed saline solution can include many reactive species such as, but not limited to at least one of O₂, H₂, Cl₂, OCl⁻, HOCl, NaOCl, HClO₂, ClO₂, HClO₃, HClO₄, H₂O₂, Na⁺, Cl⁻, H⁺, H⁻, OH⁻, O₃, O₄*⁻, 1O, OH*⁻, HOCl—O₂*⁻, HOCl—O₃, HO₂*, NaCl, HCl, NaOH, or water clusters.

In one embodiment, the at least one stable oxygen radical is a superoxide radical or “superoxide.”

Stable oxygen radicals can remain stable for about 3 months, about 6 months, about 9 months, about 12 months, about 15 months, about 18 months, about 21 months, between about 9 months and about 15 months, between about 12 months and about 18 months, at least about 9 months, at least about 12 months, at least about 15 months, at least about 18 months, about 24 months, about 30 months, about 50 months, about 100 months, or longer.

Stable oxygen radicals can be substantially stable. Substantially stable can mean that the stable oxygen radical can remain at a concentration greater than about 50% relative to the concentration on day 1, greater than about 60%, greater than about 65%, greater than about 70%, greater than about 72%, greater than about 75%, greater than about 79%, greater than about 80%, greater than about 85%, greater than about 90%, greater than about 95%, greater than about 96%, greater than about 97%, greater than about 98%, or greater than about 99% over a given time period as described above. For example, in one embodiment, the stable oxygen is at a concentration greater than about 95% relative to day 1 for at least 1 year. In another embodiment, the at least one oxygen radical is at a concentration greater than about 98% for at least 1 year.

Stability as used herein can also refer to the amount of a particular species when compared to a reference sample. In some embodiments, the reference sample can be made in 1 L vessels with 0.9% isotonic solution electrolyzed with 3 Amps at 40° F., for 3 mins. In another embodiment, the reference sample can be made according to a process as otherwise described herein. The reference standard can also be bottle directly off the processing line as a “fresh” sample.

In other embodiments, the at least one oxygen radical is greater than about 86% stable for at least 4 years, greater than about 79% stable for at least 6 years, greater than about 72% stable for at least 8 years, greater than about 65% stable for at least 10 years, or 100% stable for at least 20 years.

In some embodiments, the at least one oxygen radical is greater than about 60% stable for at least 2 years, at least 3 years, at least 4 years, at least 5 years, at least 6 years, at least 7 years, at least 8 years, at least 9 years, at least 10 years, at least 15 years, or at least 20 years. In other embodiments, the at least one oxygen radical is greater than about 65% stable for at least 2 years, at least 3 years, at least 4 years, at least 5 years, at least 6 years, at least 7 years, at least 8 years, at least 9 years, at least 10 years, at least 15 years, or at least 20 years. In still other embodiments, the at least one oxygen radical is greater than about 75% stable for at least 2 years, at least 3 years, at least 4 years, at least 5 years, at least 6 years, at least 7 years, at least 8 years, at least 9 years, at least 10 years, at least 15 years, or at least 20 years. In still other embodiments, the at least one oxygen radical is greater than about 79% stable for at least 2 years, at least 3 years, at least 4 years, at least 5 years, at least 6 years, at least 7 years, at least 8 years, at least 9 years, at least 10 years, at least 15 years, or at least 20 years. In still other embodiments, the at least one oxygen radical is greater than about 80% stable for at least 2 years, at least 3 years, at least 4 years, at least 5 years, at least 6 years, at least 7 years, at least 8 years, at least 9 years, at least 10 years, at least 15 years, or at least 20 years. In still other embodiments, the at least one oxygen radical is greater than about 90% stable for at least 2 years, at least 3 years, at least 4 years, at least 5 years, at least 6 years, at least 7 years, at least 8 years, at least 9 years, at least 10 years, at least 15 years, or at least 20 years. In still other embodiments, the at least one oxygen radical is greater than about 95% stable for at least 2 years, at least 3 years, at least 4 years, at least 5 years, at least 6 years, at least 7 years, at least 8 years, at least 9 years, at least 10 years, at least 15 years, or at least 20 years. In still other embodiments, the at least one oxygen radical is greater than about 96% stable for at least 2 years, at least 3 years, at least 4 years, at least 5 years, at least 6 years, at least 7 years, at least 8 years, at least 9 years, at least 10 years, at least 15 years, or at least 20 years. In still other embodiments, the at least one oxygen radical is greater than about 97% stable for at least 2 years, at least 3 years, at least 4 years, at least 5 years, at least 6 years, at least 7 years, at least 8 years, at least 9 years, at least 10 years, at least 15 years, or at least 20 years. In still other embodiments, the at least one oxygen radical is greater than about 98% stable for at least 2 years, at least 3 years, at least 4 years, at least 5 years, at least 6 years, at least 7 years, at least 8 years, at least 9 years, at least 10 years, at least 15 years, or at least 20 years. In still other embodiments, the at least one oxygen radical is greater than about 99% stable for at least 2 years, at least 3 years, at least 4 years, at least 5 years, at least 6 years, at least 7 years, at least 8 years, at least 9 years, at least 10 years, at least 15 years, or at least 20 years. In still other embodiments, the at least one oxygen radical is 100% stable for at least 2 years, at least 3 years, at least 4 years, at least 5 years, at least 6 years, at least 7 years, at least 8 years, at least 9 years, at least 10 years, at least 15 years, or at least 20 years.

The stability of oxygen radicals can also be stated as a decay rate over time. Substantially stable can mean a decay rate less than about 1% per month, less than about 2% per month, less than about 3% per month, less than about 4% per month, less than about 5% per month, less than about 6% per month, less than about 10% per month, less than about 3% per year, less than about 4% per year, less than about 5% per year, less than about 6% per year, less than about 7% per year, less than about 8% per year, less than about 9% per year, less than about 10% per year, less than about 15% per year, less than about 20% per year, less than about 25% per year, less than about 30% per year, less than about 40% per year, or between less than about 3% per month and less than about 7% per year.

In other embodiments, stability can be expressed as a half-life. A half-life of the stable oxygen radical can be about 6 months, about 1 year, about 2 years, about 3 years, about 4 years, about 5 years, about 10 years, about 15 years, about 20 years, about 24 years, about 30 years, about 40 years, about 50 years, greater than about 1 year, greater than about 2 years, greater than about 10 years, greater than about 20 years, greater than about 24 years, between about 1 year and about 30 years, between about 6 years and about 24 years, or between about 12 years and about 30 years.

In some embodiments, the aqueous formulations can include an electrolyzed saline solution which is a product of a circulating solution having a salt concentration of about 10.75 g NaCl/gal and electrolyzed using a set of electrodes with an amperage of about 56 amps. The set of electrodes can be arranged and numbered as desired to achieve a particular level of electrolysis. For example, two, three, four, five, six, seven, eight, nine, ten, eleven, twelve, 13, 14, 15, 16, 17, 18, 19, 20 or more electrodes can be used for electrolysis. In one embodiment, seven electrodes can be used.

Superoxide free radicals (OO*— and OOH*) and hydroxyl free radicals (OH*) can generally have a short half-life in aqueous solutions (t(½)<2 ms). In one embodiment described herein is a method to produce large-scale concentrations of these biologically active radical components in aqueous solutions that are stable and have half-lives sufficient for long-term storage. Such stable formulations also include reductive components, such that the combined formulation is of neutral pH. The produced formulations may not result in any toxicity in vitro and in vivo.

A method of production can include one or more of the steps of (1) preparation of an ultra-pure homogeneous solution of sodium chloride in water, (2) temperature control and flow regulation through a set of inert catalytic electrodes and (3) a modulated electrolytic process that results in the formation of such stable molecular moieties and complexes. In one embodiment, such a process includes all these steps.

The electro-catalytic process that forms such moieties can rely heavily on the purity and molecular homogeneity of the reactants as they make contact with the local reactive surfaces of the electrodes. Preparation of the saline solution can be a critical step in the process. The saline generally should be free from contaminants, both organic and inorganic, and homogeneous down to the molecular level. In particular, metal ions can interfere with the electro-catalytic surface reactions, and thus contamination of the water or saline by metals should be avoided.

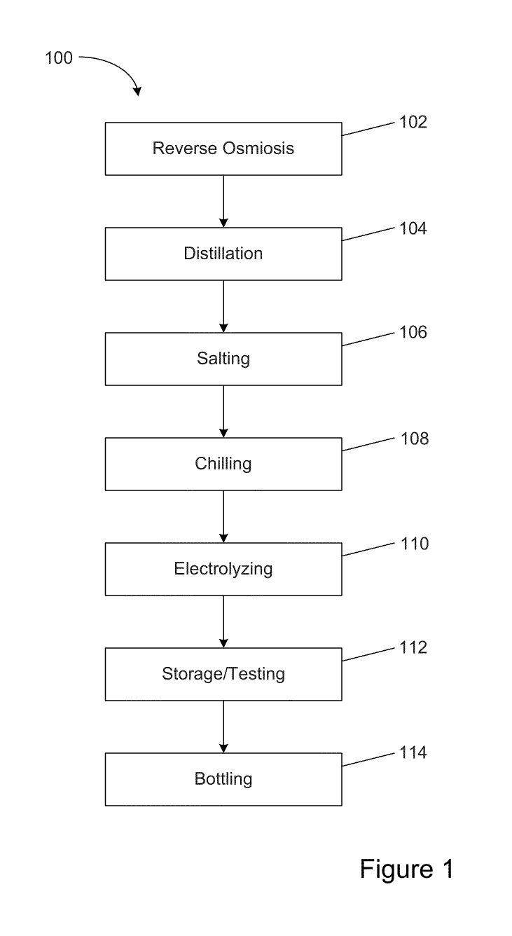

With this in mind, the first step in such a process 100 is an optional reverse osmosis procedure 102 (FIG. 1). Water can be supplied from a variety of sources, including but not limited to municipal water, filtered water, nanopure water, or the like. Municipal water, for example, can be highly variable depending on the municipal water source (e.g. stream or river versus surface or underground reservoir water), the method of sterilizing the water prior to distribution (e.g., UV light), chemicals used to treat the water, and the like. Regardless of the source of water, optionally, reverse osmosis can be used to reproducibly clean the water.

The reverse osmosis process can vary, but can provide water having a total dissolved solids content of less than about 10 ppm, about 9 ppm, about 8 ppm, about 7 ppm, about 6 ppm, about 5 ppm, about 4 ppm, about 3 ppm, about 2 ppm, about 1 ppm, 0.5 ppm, less than about 10 ppm, less than about 9 ppm, less than about 8 ppm, less than about 7 ppm, less than about 6 ppm, less than about 5 ppm, less than about 4 ppm, less than about 3 ppm, less than about 2 ppm, or less than about 1 ppm.

The temperature of the reverse osmosis process can be preformed at a temperature of about 5° C., about 10° C., about 15° C., about 20° C., about 25° C., about 30° C., or about 35° C., from about 5° C. to about 35° C., from about 10° C. to about 25° C., from about 5° C. to about 25° C., from about 10° C. to about 35° C., from about 20° C. to about 30° C., less than about 35° C., less than about 30° C., less than about 25° C., less than about 20° C., greater than about 5° C., greater than about 10° C., greater than about 15° C., or greater than about 20° C.

The process can further output cleansed water at a speed of about 1 gal/min, about 1.5 gal/min, about 2 gal/min, about 2.5 gal/min, about 3 gal/min, about 3.5 gal/min, about 4 gal/min, about 4.5 gal/min, about 5 gal/min, about 5.5 gal/min, about 6 gal/min, about 6.5 gal/min, about 7 gal/min, about 7.5 gal/min, about 8 gal/min, about 8.5 gal/min, about 9 gal/min, about 9.5 gal/min, about 10 gal/min, about 11 gal/min, or about 12 gal/min, between about 1 gal/min and about 12 gal/min, between about 2 gal/min and about 10 gal/min, between about 4 gal/min and about 8 gal/min, between about 1 gal/min and about 8 gal/min, between about 4 gal/min and about 12 gal/min, at least about 1 gal/min, at least about 2 gal/min, at least about 4 gal/min, or any range bound by any of these values.

The reverse osmosis step can be repeated as needed to achieve a particular total dissolved solids level.

Whether the optional reverse osmosis step is utilized, an optional distillation step 104 can be performed.

The distillation process can vary, but can provide water having a total dissolved solids content of less than about 5 ppm, about 4 ppm, about 3 ppm, about 2 ppm, about 1 ppm, about 0.9 ppm, about 0.8 ppm, about 0.7 ppm, about 0.6 ppm, about 0.5 ppm, about 0.4 ppm, about 0.3 ppm, about 0.2 ppm, about 0.1 ppm, less than about 1 ppm, less than about 0.9 ppm, less than about 0.8 ppm, less than about 0.7 ppm, less than about 0.6 ppm, less than about 0.5 ppm, less than about 0.4 ppm, less than about 0.3 ppm, less than about 0.2 ppm, or less than about 0.1 ppm.

The temperature of the distillation process can be preformed at a temperature of about 5° C., about 10° C., about 15° C., about 20° C., about 25° C., about 30° C., or about 35° C., from about 5° C. to about 35° C., from about 10° C. to about 25° C., from about 5° C. to about 25° C., from about 10° C. to about 35° C., from about 20° C. to about 30° C., less than about 35° C., less than about 30° C., less than about 25° C., less than about 20° C., greater than about 5° C., greater than about 10° C., greater than about 15° C., or greater than about 20° C. In one embodiment, the distillation can be run at about room temperature.

The distillation process can further output distilled water at a speed of about 250 gal/hr, about 280 gal/hr, about 300 gal/hr, about 310 gal/hr, about 320 gal/hr, about 330 gal/hr, about 335 gal/hr, about 340 gal/hr, about 345 gal/hr, about 350 gal/hr, about 355 gal/hr, about 360 gal/hr, about 365 gal/hr, about 370 gal/hr, about 375 gal/hr, about 380 gal/hr, about 385 gal/hr, about 390 gal/hr, about 395 gal/hr, about 400 gal/hr, or about 420 gal/hr, between about 340 gal/min and about 420 gal/min, between about 250 gal/min and about 365 gal/min, between about 300 gal/min and about 400 gal/min, between about 250 gal/hr and about 420 gal/hr, between about 335 gal/hr and about 385 gal/hr, at least about 250 gal/hr, at least about 280 gal/hr, at least about 300 gal/hr, or any range bound by any of these values.

The distillation step can be repeated as needed to achieve a particular total dissolved solids level. After water has been subjected to reverse osmosis, distillation, both or neither, the level of total dissolved solids in the water can be less than about 5 ppm, about 4 ppm, about 3 ppm, about 2 ppm, about 1 ppm, about 0.9 ppm, about 0.8 ppm, about 0.7 ppm, about 0.6 ppm, about 0.5 ppm, about 0.4 ppm, about 0.3 ppm, about 0.2 ppm, about 0.1 ppm, less than about 1 ppm, less than about 0.9 ppm, less than about 0.8 ppm, less than about 0.7 ppm, less than about 0.6 ppm, less than about 0.5 ppm, less than about 0.4 ppm, less than about 0.3 ppm, less than about 0.2 ppm, or less than about 0.1 ppm. The amount of total dissolved solids in the water can be an important aspect in the final product as some solids can create unwanted side products during electrolyzing. Also, unwanted solids can prevent full or efficient electrolyzing. As such, a reduction of total dissolved solids in the water, in some embodiments, is less than about 0.5 ppm.

The reverse osmosis, distillation, both or neither can be preceded by a carbon filtration system which can remove oils, alcohols, and other volatile chemical residuals and particulates that can be present in municipal water or otherwise. Also, before reverse osmosis, distillation, both or neither, water can be passed through resin tanks to remove dissolved minerals. Other methods can be used to reduce contaminants in the water such as but not limited to by deionization, carbon filtration, double-distillation, electrodeionization, resin filtration such as with Milli-Q purification, microfiltration, ultrafiltration, ultraviolet oxidation, or electrodialysis and other water purification or filtration systems.

Purified water can be used directly with the systems and methods described herein. For example, if purified water is used that has a total dissolved solids concentration of less than about 0.5 ppm, neither reverse osmosis nor distillation needs to be used. In other embodiments, if semi-purified water is used, only one of the processes may be used.

In one embodiment, contaminants can be removed from a commercial source of water by the following procedure: water flows through an activated carbon filter to remove the aromatic and volatile contaminants and then undergoes Reverse Osmosis (RO) filtration to remove dissolved solids and most organic and inorganic contaminants. The resulting filtered RO water can contain less than about 8 ppm of dissolved solids. Most of the remaining contaminants can be removed through a distillation process, resulting in dissolved solid measurements less than 1 ppm. In addition to removing contaminants, distillation may also serve to condition the water with the correct structure and Oxidation Reduction Potential (ORP) to facilitate the oxidative and reductive reaction potentials on the platinum electrodes in the subsequent electro-catalytic process.

After water has been subjected to reverse osmosis, distillation, both, neither, or a combination with another contaminant removal process, a salt is added to the water in a salting step 106. The process described herein can be applied to any ionic, soluble salt mixture, such as with those containing chlorides. The salt can be unrefined, refined, caked, de-caked, or the like. In one embodiment, the salt is sodium chloride (NaCl). Other salts can include LiCl, HCl, CuCl₂, CuSO₄, KCl, MgCl, CaCl₂. Other non-limiting examples include sulfates and phosphates. For example, strong acids such as sulfuric acid (H₂SO₄), and strong bases such as potassium hydroxide (KOH), and sodium hydroxide (NaOH) can be used as electrolytes due to their strong conducting abilities.

In some embodiments, the salt can include an additive. Salt additives can include, but are not limited to potassium iodide, sodium iodide, sodium iodate, dextrose, sodium fluoride, sodium ferrocyanide, tricalcium phosphate, calcium carbonate, magnesium carbonate, fatty acids, magnesium oxide, silicone dioxide, calcium silicate, sodium aluminosilicate, calcium aluminosilicate, ferrous fumarate, iron, or folic acid. Any of these additives can be added at this point or at any point during the described process. For example, the above additives can be added just prior to bottling.

Salt can be added to water in the form of a brine solution. Brine can be formed at a salt ratio of about 500 g NaCl/gal water, about 505 g NaCl/gal water, about 510 g NaCl/gal water, about 515 g NaCl/gal water, about 520 g NaCl/gal water, about 525 g NaCl/gal water, about 530 g NaCl/gal water, about 535 g NaCl/gal water, about 536 g NaCl/gal water, about 537 g NaCl/gal water, about 538 g NaCl/gal water, about 539 g NaCl/gal water, about 540 g NaCl/gal water, about 545 g NaCl/gal water, about 550 g NaCl/gal water, about 555 g NaCl/gal water, about 560 g NaCl/gal water, about 565 g NaCl/gal water, about 570 g NaCl/gal water, about 575 g NaCl/gal water, about 580 g NaCl/gal water, between about 500 g NaCl/gal water and about 580 g NaCl/gal water, between about 520 g NaCl/gal water and about 560 g NaCl/gal water, or between about 535 g NaCl/gal water and about 540 g NaCl/gal water. In one embodiment, the ratio can be about 537.5 g NaCl/gal water.

Brine can be formed by adding NaCl to water in a tank. For example, for a 500 gal tank, about 475 gal of water can be added to the tank and a proper amount of NaCl is added to achieve a desired ratio. The brine solution can then be thoroughly mixed for about 30 min, about 1 hr, about 6 hr, about 12 hr, about 1 day, about 2 days, about 3 days, about 4 days, about 5 days, about 6 days, about 7 days, about 8 days, about 9 days, about 10 days, or longer.

To mix the brine solution, a physical mixing apparatus can be used or a circulation or recirculation can be used. A tank can circulate or recirculate solution at a rate of about 100 gal/hr, about 200 gal/hr, about 300 gal/hr, about 400 gal/hr, about 500 gal/hr, about 600 gal/hr, about 700 gal/hr, about 800 gal/hr, about 900 gal/hr, about 1,000 gal/hr, about 1,100 gal/hr, about 1,200 gal/hr, about 1,300 gal/hr, about 1,400 gal/hr, about 1,500 gal/hr, about 1,600 gal/hr, about 1,700 gal/hr, about 1,800 gal/hr, about 1,900 gal/hr, about 2,000 gal/hr, about 2,100 gal/hr, about 2,200 gal/hr, about 2,300 gal/hr, about 2,400 gal/hr, about 2,500 gal/hr, or higher. The amount of mixing time or type of mixing used can vary. However, in some embodiments, at the end of mixing, all the salt can be dissociated.

In one embodiment, pure pharmaceutical grade sodium chloride is dissolved in the prepared distilled water to form a 15 wt % sub-saturated brine solution and continuously re-circulated and filtered until the salt has completely dissolved and all particles >0.1 microns are removed. This step can take several days. The filtered, dissolved brine solution is then injected into tanks of distilled water in about a 1:352 ratio (salt:water) in order to form a 0.3% saline solution. In one embodiment, a ratio of 10.75 g of salt per 1 gallon of water can be used to form the aqueous formulation. In another embodiment, 10.75 g of salt per 3,787.5 g of water can be used to form the aqueous formulation. This solution then can be allowed to re-circulate and diffuse until homogeneity at the molecular scale has been achieved. The diffusion coefficient of this brine in distilled water is about 1.5×10-9 m²/s at 25° C. The Einstein diffusion time (t=<x>2/2D) can then be used to determine the time it will take for the sodium chloride ions to diffuse completely in the saline solution. About 5 minutes may be required for molecules to completely diffuse 1 mm from concentration centers, and 500 minutes are required for the molecules to diffuse 1 cm based on the above approximation.

Mechanical mixing through recirculation can be required to speed diffusion. With full-tank circulation every hour for 24 hours, sodium chloride concentration centers can be homogeneous down to about the 1 cm level another 24 hours may be required to achieve diffusive homogeneity down to the molecular scale throughout the entire saline solution. The entire homogenization process can take an average of about 36 hours. Mixing discs, with microporous material, can be put in the recirculation lines to accelerate the mixing process, higher temperatures can also accelerate this process. In one embodiment, all materials and pumps that might come into contact with the saline solution can be of pristine high density hydrophobic polymer material or glass to prevent contamination. Also, tanks can remain closed to prevent atmospheric contamination.

In one embodiment, the homogenous saline solution is chilled to 4.8±0.5° C. This temperature may be critical because higher temperatures can increase reactive oxygen species (ROS) content and lower temperatures can increase hypochlorite (RS) and possibly free chlorine content during processing. Correct balance can require precisely controlled temperatures at the electro-catalytic surfaces. Careful temperature regulation during the entire electro-catalytic process is required as thermal energy generated from the electrolysis process itself may cause heating. In one embodiment, process temperatures at the electrodes can be constantly cooled and maintained at about 4.8° C. throughout electrolysis.

Brine can then be added to the previously treated water or to fresh untreated water to achieve a NaCl concentration of about 1 g NaCl/gal water, about 2 g NaCl/gal water, about 3 g NaCl/gal water, about 4 g NaCl/gal water, about 5 g NaCl/gal water, about 6 g NaCl/gal water, about 7 g NaCl/gal water, about 8 g NaCl/gal water, about 9 g NaCl/gal water, about 10 g NaCl/gal water, about 10.25 g NaCl/gal water, about 10.50 g NaCl/gal water, about 10.75 g NaCl/gal water, about 11 g NaCl/gal water, about 12 g NaCl/gal water, about 13 g NaCl/gal water, about 14 g NaCl/gal water, about 15 g NaCl/gal water, about 16 g NaCl/gal water, about 17 g NaCl/gal water, about 18 g NaCl/gal water, about 19 g NaCl/gal water, about 20 g NaCl/gal water, about 21 g NaCl/gal water, about 22 g NaCl/gal water, about 23 g NaCl/gal water, about 24 g NaCl/gal water, about 25 g NaCl/gal water, between about 1 g NaCl/gal water and about 25 g NaCl/gal water, between about 8 g NaCl/gal water and about 12 g NaCl/gal water, or between about 4 g NaCl/gal water and about 16 g NaCl/gal water.

Once brine is added to water at an appropriate amount, the solution can be thoroughly mixed for about 30 min, about 1 hr, about 6 hr, about 12 hr, about 24 hr, about 36 hr, about 48 hr, about 60 hr, about 72 hr, about 84 hr, about 96 hr, about 108 hr, about 120 hr, about 132 hr, or longer, no less than about 12 hr, no less than about 24 hr, no less than about 36 hr, no less than about 48 hr, no less than about 60 hr, no less than about 72 hr, no less than about 84 hr, no less than about 96 hr, no less than about 108 hr, no less than about 120 hr, or no less than about 132 hr.

The temperature of the liquid during mixing can be at room temperature or controlled at a temperature of about 20° C., about 25° C. about 30° C. about 35° C. about 40° C. about 45° C. about 50° C. about 55° C., about 60° C., between about 20° C. and about 40° C., between about 30° C. and about 40° C., between about 20° C. and about 30° C., between about 25° C. and about 30° C., or between about 30° C. and about 35° C.

To mix the solution, a physical mixing apparatus can be used or a circulation or recirculation can be used. A tank can circulate or recirculate solution at a rate of about 100 gal/hr, about 200 gal/hr, about 300 gal/hr, about 400 gal/hr, about 500 gal/hr, about 600 gal/hr, about 700 gal/hr, about 800 gal/hr, about 900 gal/hr, about 1,000 gal/hr, about 1,100 gal/hr, about 1,200 gal/hr, about 1,300 gal/hr, about 1,400 gal/hr, about 1,500 gal/hr, about 1,600 gal/hr, about 1,700 gal/hr, about 1,800 gal/hr, about 1,900 gal/hr, about 2,000 gal/hr, about 2,100 gal/hr, about 2,200 gal/hr, about 2,300 gal/hr, about 2,400 gal/hr, about 2,500 gal/hr, or higher. The amount of mixing time or type of mixing used can vary. In some embodiments, the mixing time is sufficient to allow complete dissociation of the NaCl.

The salt solution can then be chilled in a chilling step 108. The temperature of the chilled solution can be about 0° C., about 1° C. about 2° C. about 3° C. about 4° C. about 5° C. about 6° C. about 7° C., about 8° C., about 9° C., about 10° C., about 11° C., about 12° C., between about 0° C. and about 10° C., between about 0° C. and about 5° C., between about 5° C. and about 10° C., between about 0° C. and about 7° C., or between about 2° C. and about 5° C. In one embodiment, the chilled solution can have a temperature of between about 4.5° C. and about 5.8° C.

For large amounts of solution, various chilling and cooling methods can be employed. For example cryogenic cooling using liquid nitrogen cooling lines can be used. Likewise, the solution can be run through propylene glycol heat exchangers to achieve the desired temperature. The chilling process can take about 30 min, about 1 hr, about 2 hr, about 3 hr, about 4 hr, about 5 hr, about 6 hr, about 7 hr, about 8 hr, about 9 hr, about 10 hr, about 12 hr, about 14 hr, about 16 hr, about 18 hr, about 20 hr, about 22 hr, about 24 hr, between about 30 min and about 24 hr, between about 1 hr and about 12 hr, at least about 30 min, at least about 6 hr, at most about 24 hr, or any range created using any of these values to get the solution from room temperature to a desired chilled temperature. The chilling time can vary depending on the amount of liquid, the starting temperature and the desired chilled temperature. A skilled artisan can calculate the time required to chill a solution as described.

Products from the anodic reactions also need to be effectively transported to the cathode to provide the reactants necessary to form the stable complexes on the cathode surfaces. This requires that there be an active flow of liquid from the anode to the cathode during electrolysis. Maintaining a high degree of homogeneity in the fluids circulated between the catalytic surfaces can also be of high importance. A constant steady uniform flow of about 2-8 ml/cm²*sec can be optimal between the anode and the cathode, with typical mesh electrode distances 2 cm apart in large tanks. This flow is maintained, in part, by the convective flow of gasses released from the electrodes during electrolysis. In the small liter units, this convective flow alone can be sufficient to maintain proper circulation, in larger units, powered flow-control is necessary. Insufficient mixing or non-uniform circulation in the tanks can also cause inhomogeneities in the stream of reactants supplied to the electrodes, this will result in unpredictable and inconsistent results for the electrode reactions themselves.

For example, a build-up of excessive ROS at the electrodes can cause over-processing and build-up of undesirable reaction products in the neighborhood of the electrodes that cannot be reversed by mixing with under-processed solution elsewhere in the tank. This is also true when mixing over-processed products with under-processed products made from different tanks. For consistent product, a constant homogeneous flow of reactants should pass through the electrodes, and a consistent solution should be maintained throughout the volume of the tank. Proper flow and mixing are required. This is one of the major obstacles to scale-up the process.

The mixed solution chilled or not can then undergo electrochemical processing through the use of at least one electrode in an electrolyzing step 110. Each electrode can be or include a conductive metal. Metals can include, but are not limited to copper, aluminum, titanium, rhodium, platinum, silver, gold, iron, a combination thereof or an alloy such as steel or brass. The electrode can be coated or plated with a different metal such as, but not limited to aluminum, gold, platinum or silver. In one embodiment, each electrode is formed of titanium and plated with platinum.

In one embodiment, rough platinum-plated mesh electrodes in a vertical, coaxial, cylindrical geometry can be optimal, with not more than 2.5 cm, not more than 5 cm, not more than 10 cm, not more than 20 cm, or not more than 50 cm separation between the anode and cathode. The height of the cylindrical electrodes also can be important, as tall electrodes can promote inconsistent flow of fluids from bottom to top, as well as dissolved-oxygen gradients from bubbles generated. These factors can disrupt consistent homogeneity and uniform anode to cathode flow when comparing the bottom and top of the electrodes. Working electrodes can have a diameter of about 18 to about 25 cm, with heights not exceeding about 18 cm. Tilting the electrodes slightly may help offset the uneven-dissolved-oxygen effect. Inconsistent spacing between the electrodes can also be disruptive. Electrical current can be significantly higher between the closer-spaced surfaces of the electrodes, causing over-processing in these regions and robbing electrical current and proper reactions from surfaces that are farther apart. Inconsistencies in electrode spacing can also cause inconsistent and unpredictable results.

The amperage run through each electrode can be about 2 amps, about 3 amps, about 4 amps, about 5 amps, about 6 amps, about 7 amps, about 8 amps, about 9 amps, about 10 amps, about 11 amps, about 12 amps, about 13 amps, about 14 amps, or about 15 amps, between about 2 amps and about 15 amps, between about 4 amps and about 14 amps, at least about 2 amps, at least about 4 amps, at least about 6 amps, or any range created using any of these values. In one embodiment, 7 amps is used with each electrode.

The amperage can be run through the electrodes for a sufficient time to electrolyze the saline solution. Sufficient time can be about 1 hr, about 2 hr, about 3 hr, about 4 hr, about 5 hr, about 6 hr, about 7 hr, about 8 hr, about 9 hr, about 10 hr, about 11 hr, about 12 hr, about 13 hr, about 14 hr, about 15 hr, about 16 hr, about 17 hr, about 18 hr, about 19 hr, about 20 hr, about 21 hr, about 22 hr, about 23 hr, about 24 hr, at least about 2 hr, at least about 3 hr, at least about 4 hr, at least about 5 hr, at least about 6 hr, at least about 7 hr, at least about 8 hr, at least about 9 hr, at least about 10 hr, at least about 11 hr, at least about 12 hr, at least about 13 hr, at least about 14 hr, at least about 15 hr, at least about 16 hr, at least about 17 hr, at least about 18 hr, at least about 19 hr, at least about 20 hr, at least about 21 hr, at least about 22 hr, at least about 23 hr, at least about 24 hr, between about 2 hr and about 8 hr, between about 3 hr and about 9 hr, between about 4 hr and about 10 hr, between about 5 hr and about 12 hr, between about 7 hr and about 9 hr, between about 6 hr and about 10 hr, between about 1 hr and about 10 hr, or between about 5 hr and about 15 hr.

The solution can be chilled during the electrochemical process. The temperature during this process can be about 0° C., about 1° C. about 2° C. about 3° C. about 4° C. about 5° C. about 6° C. about 7° C., about 8° C., about 9° C., about 10° C., about 11° C., about 12° C., between about 0° C. and about 10° C., between about 0° C. and about 5° C., between about 5° C. and about 10° C., between about 0° C. and about 7° C., or between about 2° C. and about 5° C. In one embodiment, the chilled solution can have a temperature of between about 4.5° C. and about 5.8° C.

The solution can also be mixed during the electrochemical process. This mixing can be performed to ensure substantially complete electrolysis. Again, a physical mixing apparatus can be used or a circulation or recirculation can be used. Circulation or recirculation can be at a rate of about 100 gal/hr, about 200 gal/hr, about 300 gal/hr, about 400 gal/hr, about 500 gal/hr, about 600 gal/hr, about 700 gal/hr, about 800 gal/hr, about 900 gal/hr, about 1,000 gal/hr, about 1,100 gal/hr, about 1,200 gal/hr, about 1,300 gal/hr, about 1,400 gal/hr, about 1,500 gal/hr, about 1,600 gal/hr, about 1,700 gal/hr, about 1,800 gal/hr, about 1,900 gal/hr, about 2,000 gal/hr, about 2,100 gal/hr, about 2,200 gal/hr, about 2,300 gal/hr, about 2,400 gal/hr, about 2,500 gal/hr, or higher. In one embodiment, circulation or recirculation can be at about 1,000 gal/hr.

The platinum surfaces on the electrodes by themselves can be optimal to catalyze the required reactions. Rough, double layered platinum plating can assure that local “reaction centers” (sharply pointed extrusions) are active and that the reactants do not make contact with the underlying electrode titanium substrate. Tiny micropores in the platinum surface (caused by tiny bubbles in the platinum electroplating process) can allow the oxidative components to penetrate and oxidize the titanium base. This component penetration can degrade the electrodes and put undesirable titanium ions and oxides in the product. Double plated platinum can minimize the risk of micropores in the platinum surface going through to the titanium.

During electrolysis, oxygen and hydrogen bubbles themselves can form on the platinum surfaces during electrolysis and reduce the reactive surface area. Sharp, uneven surfaces can tend to minimize bubble adhesion and create stronger local electric fields, increasing efficiency.

Electric fields between the electrodes can cause movement of ions. Negative ions can move toward the anode and positive ions toward the cathode. This can enable necessary exchange of reactants and products between the electrodes. In some embodiments, no barriers are needed between the electrodes.

The configuration and electrical characteristics between the electrodes can be similar to conditions that exist between the mitochondrial membranes inside these cellular organelles. Inside living mitochondria, an electrical potential (voltage) is generated by the electron transport chain between the inner and outer mitochondrial membranes. This electrical potential is capable of causing electrolysis to take place in the mitochondria. There are many factors that regulate this voltage potential. This in principle is similar to the electrical potential maintained between the platinum electrode surfaces in the electrolysis cells. To further expand on the similarities, mitochondria produce superoxides by means of electron donation to dissolved oxygen in the cellular fluids. Similar electrochemistry can exist on the cathode of the platinum electrodes.

In the mitochondria, fluctuations of the mitochondrial potential, specifically pulsing of the potentials have been seen to take place. Pulsing potentials in the power supply of the production units can also be built in. Lack of filter capacitors in the rectified power supply can cause the voltages to drop to zero 120 times per second, resulting in a hard spike when the alternating current in the house power lines changes polarity. This hard spike, under Fourier transform, can emit a large bandwidth of frequencies. In essence, the voltage is varying from high potential to zero 120 times a second. In other embodiments, the voltage can vary from high potential to zero about 1,000 times a second, about 500 times a second, about 200 times a second, about 150 times a second, about 120 times a second, about 100 times a second, about 80 times a second, about 50 times a second, about 40 times a second, about 20 times a second, between about 200 times a second and about 20 times a second, between about 150 times a second and about 100 times a second, at least about 100 times a second, at least about 50 times a second, or at least about 120 times a second. This power modulation can allow the electrodes sample all voltages and also provides enough frequency bandwidth to excite resonances in the forming molecules themselves. The time at very low voltages can also provide an environment of low electric fields where ions of similar charge can come within close proximity to the electrodes. All of these factors together can provide a possibility for the formation of stable complexes capable of generating and preserving ROS free radicals.

Waveforms with an alternating current (AC) ripple can be used to provide power to the electrodes. Such an AC ripple can also be referred to as pulse or spiking waveforms and include: any positive pulsing currents such as pulsed waves, pulse train, square wave, sawtooth wave, pulse-width modulation (PWM), pulse duration modulation (PDM), single phase half wave rectified AC, single phase full wave rectified AC or three phase full wave rectified for example.

A bridge rectifier may be used. Other types of rectifiers can be used such as Single-phase rectifiers, Full-wave rectifiers, Three-phase rectifiers, Twelve-pulse bridge, Voltage-multiplying rectifiers, filter rectifier, a silicon rectifier, an SCR type rectifier, a high-frequency (RF) rectifier, an inverter digital-controller rectifier, vacuum tube diodes, mercury-arc valves, solid-state diodes, silicon-controlled rectifiers and the like. Pulsed waveforms can be made with a transistor regulated power supply, a dropper type power supply, a switching power supply and the like.

This pulsing waveform model can be used to stabilize superoxides, hydroxyl radicals and OOH* from many different components and is not limited to any particular variable such as voltage, amps, frequency, flux (current density) or current. The variables are specific to the components used. For example, water and NaCl can be combined which provide molecules and ions in solution. A 60 Hz current can be used, meaning that there are 60 cycles/120 spikes in the voltage (V) per second or 120 times wherein the V is 0 each second. When the V goes down to 0 it is believe that the 0 V allows for ions to drift apart/migrate and reorganize before the next increase in V. It is theorized that this spiking in V allows for and promotes a variable range of frequencies influencing many different types of compounds and/or ions so that this process occurs.

Diodes may also be used. The V may drop to 0 as many times per second as the frequency is adjusted. As the frequency is increased the number of times the V drops is increased.

When the ions are affected by the electricity from the electrodes, they change. Without being bound by theory, it is believed that the electricity alters the state of some of the ions/compounds. This alteration results in the pushing of electrons out of their original orbit and/or spin state into a higher energy state and/or a single spin state. This electrolysis provides the energy to form free radicals which are ultimately formed during a multi-generational cycling of reactants and products during the electrolysis process. In other words, compounds and/or ions are initially electrolyzed so that the products that are formed are then themselves reacted with other compounds and/or ions and/or gas to form a second generation of reactants and products. This generational process then happens again so that the products from the second generation react with other compounds and/or ions in solution when the voltage spikes again.

The redox potential can be about 840 mV.

The frequency can be from about 1 Hz to infinity or to about 100 MHz.

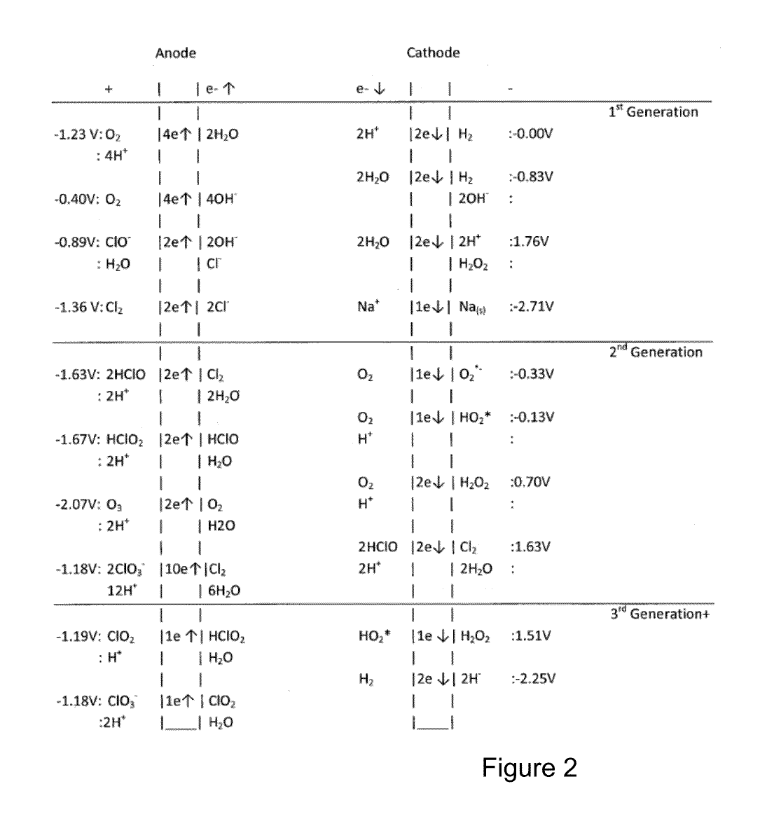

FIG. 2 illustrates an example diagram of the generation of various molecules at the electrodes, the molecules written between the electrodes depict the initial reactants and those on the outside of the electrodes depict the molecules/ions produced at the electrodes and their electrode potentials. The diagram is broken into generations where each generation relies on the products of the subsequent generations.

The end products of this electrolytic process can react within the saline solution to produce many different chemical entities. The aqueous formulations described herein can include one or more of these chemical entities. These end products can include, but are not limited to superoxides: O₂*⁻, HO₂*; hypochlorites: OCl⁻, HOCl, NaOCl; hypochlorates: HClO₂, ClO₂, HClO₃, HClO₄; oxygen derivatives: O₂, O₃, O₄*⁻, 1O; hydrogen derivatives: H₂, H⁻; hydrogen peroxide: H₂O₂; hydroxyl free Radical: OH*⁻; ionic compounds: Na⁺, Cl⁻, H⁺, OH⁻, NaCl, HCl, NaOH; chlorine: Cl₂; and water clusters: n*H₂O-induced dipolar layers around ions, several variations.

In order to determine the relative concentrations and rates of production of each of these during electrolysis, certain general chemical principles can be helpful:

1) A certain amount of Gibbs free energy is required for construction of the molecules; Gibbs free energy is proportional to the differences in electrode potentials listed in FIG. 2. Reactions with large energy requirements are less likely to happen, for example an electrode potential of −2.71V (compared to Hydrogen reduction at 0.00V) is required to make sodium metal:

Na⁺+1e⁻→Na₍ₛ₎

Such a large energy difference requirement makes this reaction less likely to happen compared to other reactions with smaller energy requirements. Electron(s) from the electrodes may be preferentially used in the reactions that require lesser amounts of energy, such as the production of hydrogen gas.

2) Electrons and reactants are required to be at the same micro-locality on the electrodes. Reactions that require several reactants may be less likely to happen, for example:

Cl₂+6H₂O→10e⁻+2ClO₃⁻+12H⁺

requires that 6 water molecules and a Cl₂ molecule to be at the electrode at the same point at the same time and a release of 10 electrons to simultaneously occur. The probability of this happening generally is smaller than other reactions requiring fewer and more concentrated reactants to coincide, but such a reaction may still occur.

3) Reactants generated in preceding generations can be transported or diffuse to the electrode where reactions happen. For example, dissolved oxygen (O₂) produced on the anode from the first generation can be transported to the cathode in order to produce superoxides and hydrogen peroxide in the second generation. Ions can be more readily transported: they can be pulled along by the electric field due to their electric charge. In order for chlorates, to be generated, for example, HClO₂ can first be produced to start the cascade, restrictions for HClO₂ production can also restrict any subsequent chlorate production. Lower temperatures can prevent HClO₂ production.

Stability and concentration of the above products can depend, in some cases substantially, on the surrounding environment. The formation of complexes and water clusters can affect the lifetime of the moieties, especially the free radicals.

In a pH-neutral aqueous solution (pH around 7.0) at room temperature, superoxide free radicals (O₂*⁻) have a half-life of 10's of milliseconds and dissolved ozone (O₃) has a half-life of about 20 min. Hydrogen peroxide (H₂O₂) is relatively long-lived in neutral aqueous environments, but this can depend on redox potentials and UV light. Other entities such as HCl and NaOH rely on acidic or basic environments, respectively, in order to survive. In pH-neutral solutions, H⁺ and OH⁻ ions have concentrations of approximately 1 part in 10,000,000 in the bulk aqueous solution away from the electrodes. H⁻ and ¹O can react quickly. The stability of most of these moieties mentioned above can depend on their microenvironment. Superoxides and ozone can form stable Van de Waals molecular complexes with hypochlorites. Clustering of polarized water clusters around charged ions can also have the effect of preserving hypochlorite-superoxide and hypochlorite-ozone complexes. Such complexes can be built through electrolysis on the molecular level on catalytic substrates, and may not occur spontaneously by mixing together components. Hypochlorites can also be produced spontaneously by the reaction of dissolved chlorine gas (Cl₂) and water. As such, in a neutral saline solution the formation of one or more of the stable molecules and complexes may exist: dissolved gases: O₂, H₂, Cl₂; hypochlorites: OCl⁻, HOCl, NaOCl; hypochlorates: HClO₂, ClO₂, HClO₃, HClO₄; hydrogen peroxide: H₂O₂; ions: Na⁺, Cl⁻, H⁺, H⁻, OH⁻; ozone: O₃, O₄*⁻; singlet oxygen: ¹O; hydroxyl free radical: OH*⁻; superoxide complexes: HOCl—O₂*⁻; and ozone complexes: HOCl—O₃. One or more of the above molecules can be found within the aqueous formulations described herein.

A complete quantum chemical theory can be helpful because production is complicated by the fact that different temperatures, electrode geometries, flows and ion transport mechanisms and electrical current modulations can materially change the relative/absolute concentrations of these components, which could result in producing different distinct compositions. As such, the selection of production parameters can be critical. The amount of time it would take to check all the variations experimentally may be prohibitive.

After amperage has been run through the solution for a sufficient time, an electrolyzed solution is created with stable reactive species. The solution can have a pH of about 7.4, about 7.5, about 7.6, about 7.7, about 7.8, about 7.9, about 8.0, about 8.1, about 8.2 about 8.3, about 8.4, between about 7.4 and about 8.4, or between about 8.0 and 8.2. In one embodiment, the pH is about 8.01. In some embodiments, the pH is greater than 7.4. In some embodiments, the pH is not acidic. In other embodiments, the solution can have a pH less than about 7.5. The pH may not be basic. The solution can be stored and or tested for particular properties in storage/testing step 112.

The chlorine concentration of the electrolyzed solution can be about 5 ppm, about 10 ppm, about 15 ppm, about 20 ppm, about 21 ppm, about 22 ppm, about 23 ppm, about 24 ppm, about 25 ppm, about 26 ppm, about 27 ppm, about 28 ppm, about 29 ppm, about 30 ppm, about 31 ppm, about 32 ppm, about 33 ppm, about 34 ppm, about 35 ppm, about 36 ppm, about 37 ppm, about 38 ppm, less than about 38 ppm, less than about 35 ppm, less than about 32 ppm, less than about 28 ppm, less than about 24 ppm, less than about 20 ppm, less than about 16 ppm, less than about 12 ppm, less than about 5 ppm, between about 30 ppm and about 34 ppm, between about 28 ppm and about 36 ppm, between about 26 ppm and about 38 ppm, between about 20 ppm and about 38 ppm, between about 5 ppm and about 34 ppm, between about 10 ppm and about 34 ppm, or between about 15 ppm and about 34 ppm. In one embodiment, the chlorine concentration is about 32 ppm. In another embodiment, the chlorine concentration is less than about 41 ppm.

The saline concentration in the electrolyzed solution can be about 0.10% w/v, about 0.11% w/v, about 0.12% w/v, about 0.13% w/v, about 0.14% w/v, about 0.15% w/v, about 0.16% w/v, about 0.17% w/v, about 0.18% w/v, about 0.19% w/v, about 0.20% w/v, about 0.30% w/v, about 0.40% w/v, about 0.50% w/v, about 0.60% w/v, about 0.70% w/v, between about 0.10% w/v and about 0.20% w/v, between about 0.11% w/v and about 0.19% w/v, between about 0.12% w/v and about 0.18% w/v, between about 0.13% w/v and about 0.17% w/v, or between about 0.14% w/v and about 0.16% w/v.

The aqueous formulation generally can include electrolytic and/or catalytic products of pure saline that mimic redox signaling molecular compositions of the native salt water compounds found in and around human cells. The aqueous formulation can be fine tuned to mimic or mirror molecular compositions of different biological media. The aqueous formulation can have reactive species other than chlorine present. As described, species present in the compositions and aqueous formulations described herein can include, but are not limited to O₂, H₂, Cl₂, OCl⁻, HOCl, NaOCl, HClO₂, ClO₂, HClO₃, HClO₄, H₂O₂, Na⁺, Cl⁻, H⁺, H⁻, OH⁻, O₃, O₄*⁻, ¹O, OH*⁻, HOCl—O₂*⁻, HOCl—O₃, O₂*⁻, HO₂*, NaCl, HCl, NaOH, and water clusters: n*H₂O-induced dipolar layers around ions, several variations.

Depending on the parameters used to produce the aqueous formulation, different components can be present at different concentrations. In one embodiment, the aqueous formulation can include about 0.1 ppt, about 0.5 ppt, about 1 ppt, about 1.5 ppt, about 2 ppt, about 2.5 ppt, about 3 ppt, about 3.5 ppt, about 4 ppt, about 4.5 ppt, about 5 ppt, about 6 ppt, about 7 ppt, about 8 ppt, about 9 ppt, about 10 ppt, about 20 ppt, about 50 ppt, about 100 ppt, about 200 ppt, about 400 ppt, about 1,000 ppt, between about 0.1 ppt and about 1,000 ppt, between about 0.1 ppt and about 100 ppt, between about 0.1 ppt and about 10 ppt, between about 2 ppt and about 4 ppt, at least about 0.1 ppt, at least about 2 ppt, at least about 3 ppt, at most about 10 ppt, or at most about 100 ppt of OCl⁻. In some embodiments, OCl⁻ can be present at about 3 ppt. In other embodiments, OCl⁻ can be the predominant chlorine containing species in the aqueous formulation.

In some embodiments, hydroxyl radicals can be stabilized in the aqueous formulation by the formation of radical complexes. The radical complexes can be held together by hydrogen bonding. Another radical that can be present in the aqueous formulation is an OOH. radical. Still other radical complexes can include a nitroxyl-peroxide radical (HNO—HOO*) and/or a hypochlorite-peroxide radical (HOCl—HOO*).

Reactive species' concentrations in the solutions, detected by fluorescence photo spectroscopy, may not significantly decrease in time. Mathematical models show that bound HOCl—*O₂⁻ complexes are possible at room temperature. Molecular complexes can preserve volatile components of reactive species. For example, reactive species concentrations in whole blood as a result of molecular complexes may prevent reactive species degradation over time.

Reactive species can be further divided into “reduced species” (RS) and ROS. Reactive species can be formed from water molecules and sodium chloride ions when restructured through a process of forced electron donation. Electrons from lower molecular energy configurations in the salinated water may be forced into higher, more reactive molecular configurations. The species from which the electron was taken can be “electron hungry” and is called the RS and can readily become an electron acceptor (or proton donor) under the right conditions. The species that obtains the high-energy electron can be an electron donor and is called the ROS and may energetically release these electrons under the right conditions.

When an energetic electron in ROS is unpaired it is called a “radical”. ROS and RS can recombine to neutralize each other by the use of a catalytic enzyme. Three elements, (1) enzymes, (2) electron acceptors, and (3) electron donors can all be present at the same time and location for neutralization to occur.

In some embodiments, substantially no organic material is present in the aqueous formulations described. Substantially no organic material can be less than about 0.1 ppt, less than about 0.01 ppt, less than about 0.001 ppt or less than about 0.0001 ppt of total organic material.

The aqueous formulation can be stored and bottled or placed in suitable containers as needed to ship to consumers. The aqueous formulation can have a shelf life of about 5 days, about 30 days, about 3 months, about 6 months, about 9 months, about 1 year, about 1.5 years, about 2 years, about 3 years, about 5 years, about 10 years, at least about 5 days, at least about 30 days, at least about 3 months, at least about 6 months, at least about 9 months, at least about 1 year, at least about 1.5 years, at least about 2 years, at least about 3 years, at least about 5 years, at least about 10 years, between about 5 days and about 1 year, between about 5 days and about 2 years, between about 1 year and about 5 years, between about 90 days and about 3 years, between about 90 days and about 5 year, or between about 1 year and about 3 years.

The aqueous formulation can then be bottled in a bottling step 114. The aqueous formulation can be bottled in non-reactive bottles, vessels, pouches, or containers. In one embodiment, the aqueous formulation can be bottled in polymeric or plastic bottles or containers having volumes of about 4 oz, about 8 oz, about 16 oz, about 32 oz, about 48 oz, about 64 oz, about 80 oz, about 96 oz, about 112 oz, about 128 oz, about 144 oz, about 160 oz, or any range created using any of these values. Polymeric or plastic materials can include but are not limited to polyethylene, polypropylene, polystyrene, and the like.

Further, the aqueous formulation can be bottled in non-polymeric containers. Non-polymeric materials can include ceramic, glass, clay, and the like. In some embodiments, the non-polymeric containers can be non-reactive. In one embodiment, the container is a borosilicate glass bottle. A glass container can be colored or non-colored to reduce interaction with light. Non-polymeric bottles can have volumes of about 4 oz, about 8 oz, about 16 oz, about 32 oz, about 48 oz, about 64 oz, about 80 oz, about 96 oz, about 112 oz, about 128 oz, about 144 oz, about 160 oz, or any range created using any of these values.

The containers can also be squeezable pouches having similar volumes. In one embodiment, squeezable pouches can have one way valves to prevent leakage of the aqueous formulation, for example, during athletic activity.

During bottling, solution from an approved batch can be pumped through a 10 micron filter (e.g., polypropylene) to remove any larger particles from tanks, dust, hair, etc. that might have found their way into the batch. In other embodiments, this filter need not be used. Then, the solution can be pumped into the bottles, the overflow going back into the batch.

Containers or bottles generally may not contain any dyes, metal specks or chemicals that can be dissolved by acids or oxidating agents. The bottles, caps, bottling filters, valves, lines and heads used can be specifically rated for acids and oxidating agents. Caps and with organic glues, seals or other components sensitive to oxidation may be avoided, as these could neutralize and weaken the product over time.

The containers, vessels, bottles and pouches used herein can aid in preventing decay of free radical species found within the aqueous formulations. In other embodiments, the bottles and pouches described do not further the decay process. In other words, the containers, vessels, bottles and pouches used can be inert with respect to the radical species in the aqueous formulations. In one embodiment, a container (e.g., bottle and/or pouch) can allow less than about 10% decay/month, less than about 9% decay/month, less than about 8% decay/month, less than about 7% decay/month, less than about 6% decay/month, less than about 5% decay/month, less than about 4% decay/month, less than about 3% decay/month, less than about 2% decay/month, less than about 1% decay/month, between about 10% decay/month and about 1% decay/month, between about 5% decay/month and about 1% decay/month, about 10% decay/month, about 9% decay/month, about 8% decay/month, about 7% decay/month, about 6% decay/month, about 5% decay/month, about 4% decay/month, about 3% decay/month, about 2% decay/month, or about 1% decay/month of free radicals in the aqueous formulation. In one embodiment, a container, vessel, bottles or pouch can only result in about 3% decay/month of superoxide. In another embodiment, a pouch can only result in about 4% decay/month of superoxide.

Quality Assurance testing can be done on every batch before the batch can be approved for bottling or can be performed during or after bottling. A 16 oz. sample bottle can be taken from each complete batch and analyzed. Determinations for presence of contaminants such as heavy metals or chlorates can be performed. Then pH, Free and Total Chlorine concentrations and reactive molecule concentrations of the active ingredients can be analyzed by fluorospectroscopy methods. These results can be compared to those of a standard solution which is also tested along side every sample. If the results for the batch fall within a certain range relative to the standard solution, it can be approved. A chemical chromospectroscopic mass spectrometer (MS) analysis can also be run on random samples to determine if contaminants from the production process are present.

The aqueous formulation can be consumed by ingestion. In other embodiments, the aqueous formulation can be provided as a solution for injection. In some embodiments, injection can be subcutaneous, intra-luminal, site specific (e.g., injected into a cancer or internal lesion), or intramuscular. Intravenous injection can also be desirable. The aqueous formulation can be packaged in plastic medical solution pouches having volumes of about 4 oz, about 8 oz, about 16 oz, about 32 oz, about 48 oz, about 64 oz, about 80 oz, about 96 oz, about 112 oz, about 128 oz, about 144 oz, about 160 oz, or any range created using any of these values, and these pouches can be used with common intravenous administration systems.

When administered as a liquid aqueous formulation, it can be taken once, twice, three times, four times or more a day. Each administration can be about 1 oz, about 2 oz, about 3 oz, about 4 oz, about 5 oz, about 6 oz, about 7 oz, about 8 oz, about 9 oz, about 10 oz, about 11 oz, about 12 oz, about 16 oz, about 20 oz, about 24 oz, about 28 oz, about 32 oz, about 34 oz, about 36 oz, about 38 oz, about 40 oz, about 46 oz, between about 1 oz and about 32 oz, between about 1 oz and about 16 oz, between about 1 oz and about 8 oz, at least about 2 oz, at least about 4 oz, or at least about 8 oz. In one embodiment, the aqueous formulation can be administered at a rate of about 4 oz twice a day.

In other embodiments, the administration can be acute or long term. For example, the aqueous formulation can be consumed for a day, a week, a month, a year or longer. In other embodiments, the aqueous formulation can simply be taken as needed such as for exercise.

The aqueous formulations described herein when administered can be used to treat a condition or a disease or can enhance a life condition or a condition associated with a disease. For example, when administered alongside exercise, the aqueous formulations described herein can increase the density of mitochondrial DNA. For example, an increase in mitochondrial DNA of about 1%, about 5%, about 10%, about 15%, about 20%, about 21%, about 22%, about 23%, about 24%, about 25%, about 26%, about 27%, about 28%, about 29%, about 30%, about 32%, about 34%, about 36%, about 38%, about 40%, about 45%, between about 1% and about 40%, between about 1% and about 10%, between about 20% and about 30%, at least about 5%, at least about 10%, or at least about 20% when compared to an individual who has not taken the aqueous formulation. An increase in mitochondrial DNA can result in a lower level of free radicals in the blood which can in turn lead to a reduced amount of oxidative stress.

An increase in mitochondrial DNA can be used to treat a condition or a disease or can enhance a life condition or a condition associated with a disease. As such, the aqueous formulations described can treat conditions or diseases such as, but not limited to sacropenia, Parkinson's disease, neuro-related age disease, obesity, aging, life stresses such as those caused by fear, neurodegenerative diseases, cognitive disorders, obesity, reduced metabolic rate, metabolic syndrome, diabetes mellitus, cardiovascular disease, hyperlipidemia, neurodegenerative disease, cognitive disorder, mood disorder, stress, and anxiety disorder; for weight management, or to increase muscle performance or mental performance, AIDS, dementia complex, Alzheimer's disease, amyotrophic lateral sclerosis, adrenoleukodystrophy, Alexander disease, Alper's disease, ataxia telangiectasia, Batten disease, bovine spongiform encephalopathy (BSE), Canavan disease, corticobasal degeneration, Creutzfeldt-Jakob disease, dementia with Lewy bodies, fatal familial insomnia, frontotemporal lobar degeneration, Huntington's disease, Kennedy's disease, Krabbe disease, Lyme disease, Machado-Joseph disease, multiple sclerosis, multiple system atrophy, neuroacanthocytosis, Niemann-Pick disease, Pick's disease, primary lateral sclerosis, progressive supranuclear palsy, Refsum disease, Sandhoff disease, diffuse myelinoclastic sclerosis, spinocerebellar ataxia, subacute combined degeneration of spinal cord, tabes dorsalis, Tay-Sachs disease, toxic encephalopathy, transmissible spongiform encephalopathy, and wobbly hedgehog syndrome, cognitive function abnormalities, perception abnormalities, attention disorders, speech comprehension disorders, reading comprehension disorders, creation of imagery disorders, learning disorders, reasoning disorders, mood disorders, depression, postpartum depression, dysthymia, bipolar disorder, generalized anxiety disorder, panic disorder, panic disorder with agoraphobia, agoraphobia, social anxiety disorder, obsessive-compulsive disorder, post-traumatic stress disorder, musculoskeletal disorder, lack of strength, lack of endurance, cancer, atherosclerotic lesions, atherosclerosis, oxidative stress, atherogenesis, hypertension, hypercholesterolemia, and degenerative diseases.

The aqueous formulations described herein when administered can be used to increase athletic performance. The aqueous formulations can increase athletic performance, for example, when taken before or concurrently with exercise, one can increase their time to exhaustion. For example, when using the aqueous formulation, one can increase their time to exhaustion by about 1%, about 5%, about 10%, about 15%, about 20%, about 21%, about 22%, about 23%, about 24%, about 25%, about 26%, about 27%, about 28%, about 29%, about 30%, about 32%, about 34%, about 36%, about 38%, about 40%, about 45%, between about 1% and about 40%, between about 1% and about 10%, between about 20% and about 30%, at least about 5%, at least about 10%, or at least about 20% when compared to an individual who has not taken the aqueous formulation.

Also, the aqueous formulation can increase a recipient's VO₂ₘₐₓ. The aqueous formulation can contain signaling molecules. Within 30 minutes of drinking, small molecules form, shifting the metabolome. Athletes drinking the aqueous formulation for one week or longer can experience a shift in up to 43 metabolites, such as energy intermediates. People, and even animals, treated with ASEA for one week ran 29% longer until exhausted. Muscle and liver glycogen can be modulated by administration of ASEA. Muscle fatty acid β-oxidation can be modulated by administration of ASEA. Muscle carbonyls can be modulated by administration of ASEA.

The aqueous formulations described can contain stabilized superoxides, hydroxyl radicals and OOH* which can have many industrial uses as well, such as degrading plastics into organic components, leaching metals and as a color-safe bleach.

### Example 1

### Large Scale Manufacture

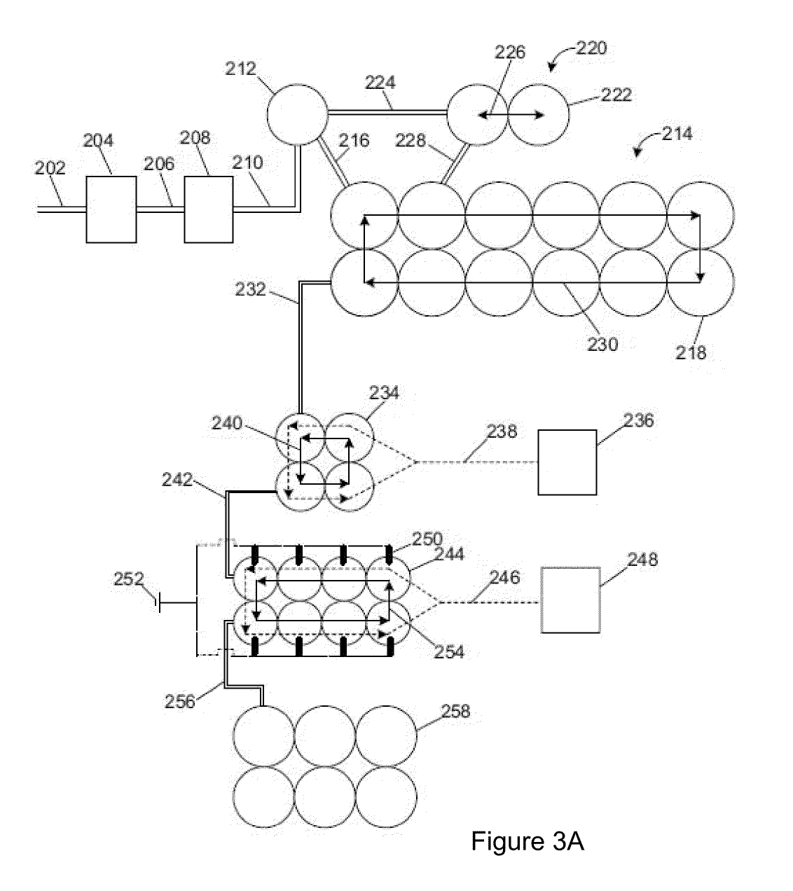

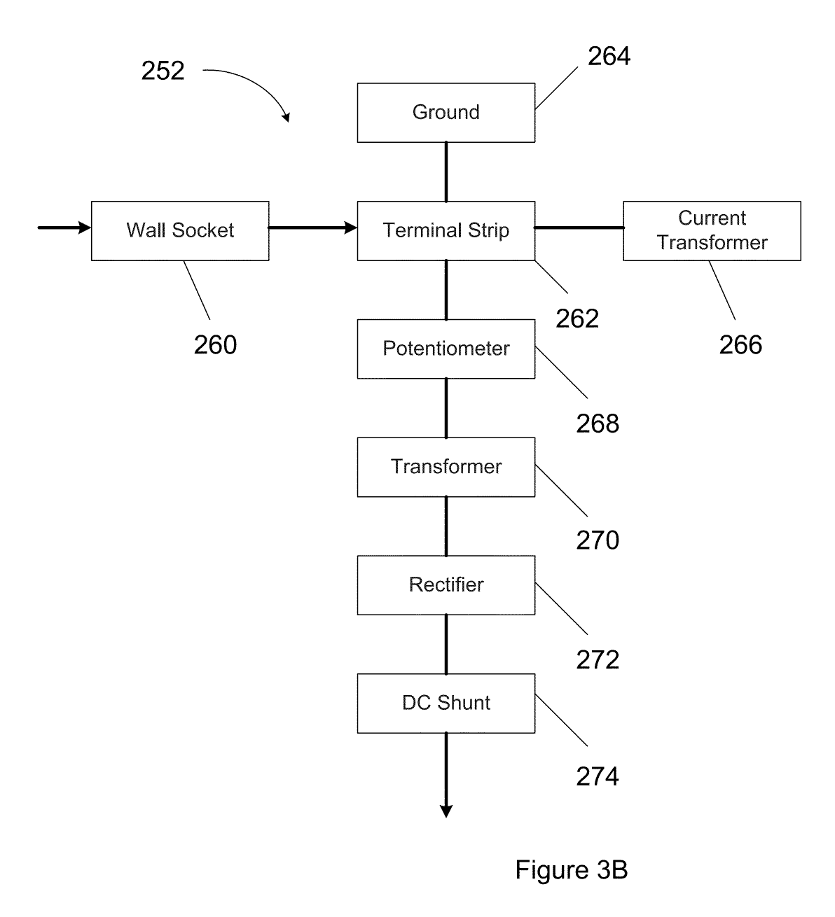

FIG. 3A illustrates a plan view of a process and system for producing an aqueous formulation according to the present description. One skilled in the art understands that changes can be made to the system to alter the aqueous formulation, and these changes are within the scope of the present description.

Incoming water 202 can be subjected to reverse osmosis system 204 at a temperature of about 15-20° C. to achieve purified water 206 with about 8 ppm of total dissolves solids. Purified water 206, is then fed at a temperature of about 15-20° C. into distiller 208 and processed to achieve distilled water 210 with about 0.5 ppm of total dissolved solids. Distilled water 210 can then be stored in tank 212.

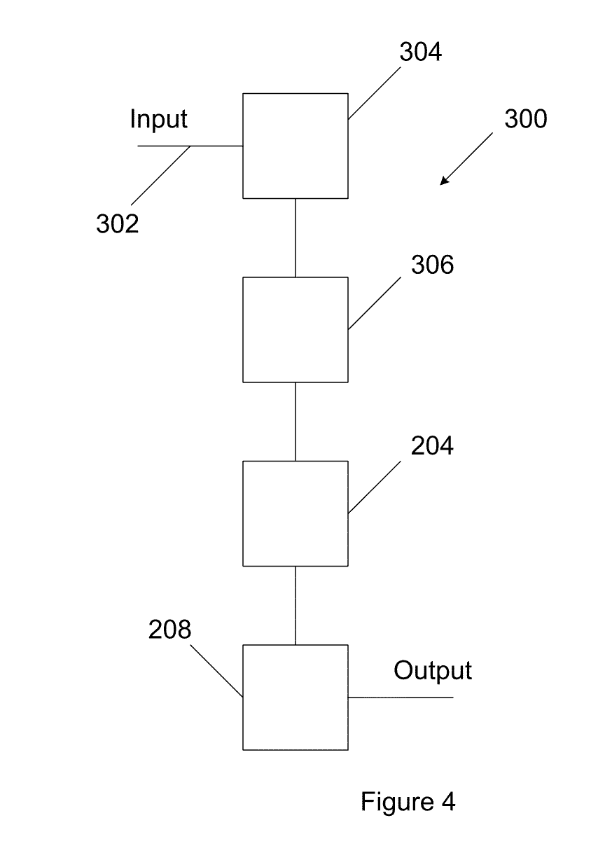

FIG. 4 illustrates an example system for preparing water for further processing into an aqueous formulation. System 300 can include a water source 302 which can feed directly into a carbon filter 304. After oils, alcohols, and other volatile chemical residuals and particulates are removed by carbon filter 304, the water can be directed to resin beds within a water softener 306 which can remove dissolved minerals. Then, as described above, the water can pass through reverse osmosis system 204 and distiller 208.

As needed, distilled water 210 can be gravity fed from tank 212 into saline storage tank cluster 214 using line 216. Saline storage tank cluster 214 in one embodiment can include twelve tanks 218. Each tank 218 can be filled to about 1,300 gallons with distilled water 210. A handheld meter can be used to test distilled water 210 for salinity.

Saline storage tank cluster 214 is then salted using a brine system 220. Brine system 220 can include two brine tanks 222. Each tank can have a capacity of about 500 gallons. Brine tanks 222 are filled to 475 gallons with distilled water 210 using line 224 and then NaCl is added to the brine tanks 222 at a ratio of about 537.5 g/gal of liquid. At this point, the water is circulated 226 in the brine tanks 222 at a rate of about 2,000 gal/hr for about 4 days.

Prior to addition of brine to tanks 218, the salinity of the water in tanks 218 can be tested using a handheld conductivity meter such as an YSI ECOSENSE® ecp300 (YSI Inc., Yellow Springs, Ohio). Any corrections based on the salinity measurements can be made at this point. Brine solution 228 is then added to tanks 218 to achieve a salt concentration of about 10.75 g/gal. The salted water is circulated 230 in tanks 218 at a rate of about 2,000 gal/hr for no less than about 72 hr. This circulation is performed at room temperature. A handheld probe can again be used to test salinity of the salinated solution. In one embodiment, the salinity is about 2.8 ppth.

In one example, the method for filling and mixing the salt water in the brine holding tanks, the amount of liquid remaining in the tanks is measured. The amount of liquid remaining in a tank is measured by recording the height that the liquid level is from the floor that sustains the tank, in centimeters, and referencing the number of gallons this height represents. This can be done from the outside of the tank if the tank is semi-transparent. The initial liquid height in both tied tanks can also be measured. Then, after ensuring that the output valve is closed, distilled water can be pumped in. The amount of distilled water that is being pumped into a holding tank can then be calculated by measuring the rise in liquid level: subtracting the initial height from the filled height and then multiplying this difference by a known factor.

The amount of salt to be added to the tank is then calculated by multiplying 11 grams of salt for every gallon of distilled water that has been added to the tank. The salt can be carefully weighed out and dumped into the tank.

The tank is then agitated by turning on the recirculation pump and then opening the top and bottom valves on the tank. Liquid is pumped from the bottom of the tank to the top. The tank can be agitated for three days before it may be ready to be processed.

After agitating the tank for more than 6 hours, the salinity is checked with a salinity meter by taking a sample from the tank and testing it. Salt or water can be added to adjust the salinity within the tanks. If either more water or more salt is added then the tanks are agitated for 6 more hours and tested again. After about three days of agitation, the tank is ready to be processed.

Salinated water 232 is then transferred to cold saline tanks 234. In one embodiment, four 250 gal tanks are used. The amount of salinated water 232 moved is about 1,000 gal. A chiller 236 such as a 16 ton chiller is used to cool heat exchangers 238 to about 0-5° C. The salinated water is circulated 240 through the heat exchangers which are circulated with propylene glycol until the temperature of the salinated water is about 4.5-5.8° C. Chilling the 1,000 gal of salinated water generally takes about 6-8 hr.

Cold salinated water 242 is then transferred to processing tanks 244. In one embodiment, eight tanks are used and each can have a capacity of about 180 gal. Each processing tank 244 is filled to about 125 gal for a total of 1,000 gal. Heat exchangers 246 are again used to chill the cold salinated water 242 added to processing tanks 244. Each processing tank can include a cylinder of chilling tubes and propylene glycol can be circulated. The heat exchangers can be powered by a 4-5 ton chiller 248. The temperature of cold salinated water 242 can remain at 4.5-5.8° C. during processing.

Prior to transferring aged salt water to processing tanks, the aged salt water can be agitated for about 30 minutes to sufficiently mix the aged salt water. Then, the recirculation valves can be closed, the appropriate inlet valve on the production tank is opened, and the tank is filled so that the salt water covers the cooling coils and comes up to the fill mark (approximately 125 gallons).

Once the aged salt water has reached production temperature, the recirculation pump is turned off but the chiller is left on. The tank should be adequately agitated or re-circulated during the whole duration of electrochemical processing and the temperature should remain constant throughout.

Each processing tank 244 includes electrodes 250. Electrodes 250 can be 3 inches tall circular structures formed of titanium and plated with platinum. Electrochemical processing of the cold salinated water can be run for 8 hr. A power supply 252 is used to power the eight electrodes (one in each processing tank 244) to 7 amps each for a total of 56 amps. The cold salinated water is circulated 254 during electrochemical processing at a rate of about 1,000 gal/hr.

An example power supply is illustrated in FIG. 3B. Power supply 252 includes a power input such as readily available electricity from such as from a wall socket 260. Wall socket can provide any type of AC or DC power. The wall plug feeds into a terminal strip 262, also known as a terminal block. Terminal strip 262 acts like a surge protector allowing a number of electrical connections from the strip to other devices. For example, terminal strip 262 can be an interface for electrical circuits. Terminal strip 262 can be connected to ground 264 and/or current transformer 266. A transformer can be used to measure electric currents. Terminal strip 262 can also be connected to potentiometer 268 which can measure voltage across an electrical system and can be used to aid in adjusting the voltage. For example a dial can be connected to potentiometer 268 so that the operator may adjust the voltage as desired.

A second transformer 270 can be connected to potentiometer 268, which can then be operably connected to rectifier 272. Rectifiers in general convert alternating current (AC) to direct current (DC). In one embodiment, rectifier 272 is a bridge rectifier. Converting the waveform into one with a constant polarity can increase the voltage output. This waveform is called a full wave rectified signal. Once the waveform and voltage are configured as desired, DC shunt 274 can provide a means for bringing electricity to different devices such as electrodes, monitors and other operational instruments. In one embodiment, the DC shunt provides electricity to the electrodes described herein. A DC shunt is an optional feature in a power supply as described and may or may not be used.

In some embodiments, one transformer can be used in a power supply. In other embodiments, more than two transformers can be used, for example, three, four, five six or more transformers can be used.

An independent current meter can be used to set the current to around 7.0 Amps. Attention can be paid to ensure that the voltage does not exceed 12V and does not go lower than 9V. Normal operation can be about 10V.

A run timer can be set for a prescribed time (about 4.5 to 5 hours). Each production tank can have its own timer and/or power supply. Electrodes should be turned off after the timer has expired.

The production tanks can be checked periodically. The temperature and/or electrical current can be kept substantially constant. At the beginning, the electrodes can be visible from the top, emitting visible bubbles. After about 3 hours, small bubbles of un-dissolved oxygen can start building up in the tank as oxygen saturation occurs, obscuring the view of the electrodes. A slight chlorine smell can be normal.

After the 8 hour electrochemical processing is complete, aqueous formulation 256 has been created with a pH of about 7.4, 32 ppm of chlorine. Aqueous formulation 256 is transferred to storage tanks 258 where the aqueous formulation awaits bottling and is shipped to consumers as an aqueous formulation.

### Example 2

### Characterization of a Beverage Produced as Described in Example 1

A beverage produced as described in Example 1 and marketed under the trade name ASEA® was analyzed using a variety of different characterization techniques. ICP/MS and 35Cl NMR were used to analyze and quantify chlorine content. Headspace mass spectrometry analysis was used to analyze adsorbed gas content in the beverage. 1H NMR was used to verify the organic matter content in the beverage. 31P NMR and EPR experiments utilizing spin trap molecules were used to explore the beverage for free radicals.

The beverage was received and stored at about 4° C. when not being used.

### Chlorine NMR

Sodium hypochlorite solutions were prepared at different pH values. 5% sodium hypochlorite solution had a pH of 12.48. Concentrated nitric acid was added to 5% sodium hypochlorite solution to create solutions that were at pH of 9.99, 6.99, 5.32, and 3.28. These solutions were then analyzed by NMR spectroscopy. The beverage had a measured pH of 8.01 and was analyzed directly by NMR with no dilutions.

NMR spectroscopy experiments were performed using a 400 MHz Bruker spectrometer equipped with a BBO probe. Cl35 NMR experiments were performed at a frequency of 39.2 MHz using single pulse experiments. A recycle delay of 10 seconds was used, and 128 scans were acquired per sample. A solution of NaCl in water was used as an external chemical shift reference. All experiments were performed at room temperature.

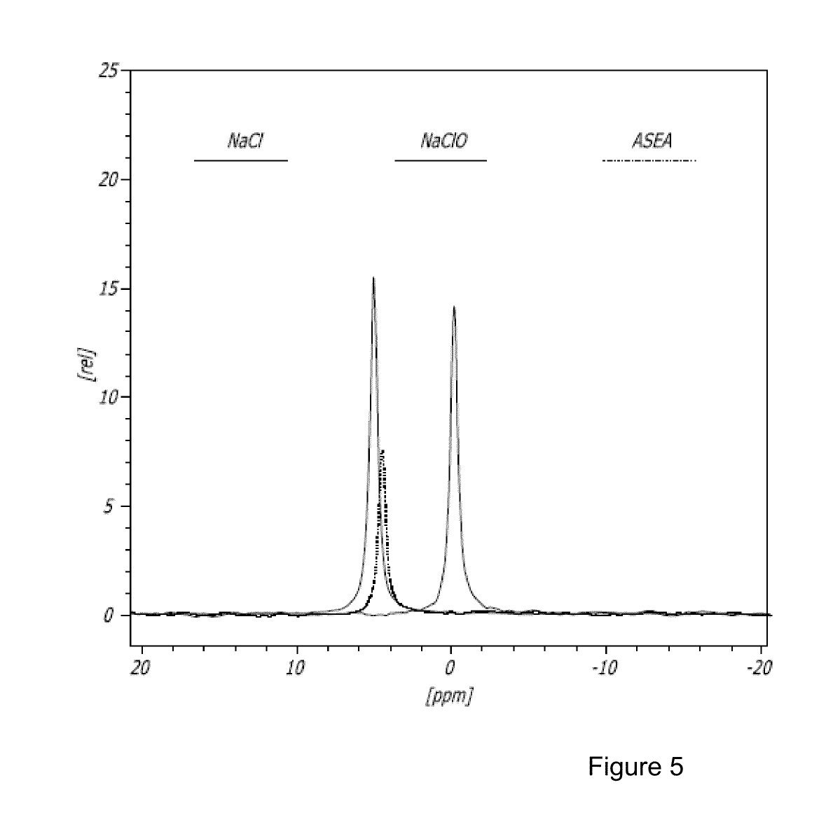

Cl35 NMR spectra were collected for NaCl solution, NaClO solutions adjusted to different pH values, and the beverage. FIG. 5 shows the Cl35 spectra of NaCl, NaClO solution at a pH of 12.48, and the beverage. The chemical shift scale was referenced by setting the Cl⁻ peak to 0 ppm. NaClO solutions above a pH of 7 had identical spectra with a peak at approximately 5.1 ppm. Below pH of 7.0, the ClO⁻ peak disappeared and was replaced by much broader, less easily identifiable peaks. The beverage was presented with one peak at approximately 4.7 ppm, from ClO⁻ in the beverage solution. This peak was integrated to estimate the concentration of ClO⁻ in the beverage solution, which was determined to be 2.99 ppt or 0.17 M of ClO⁻ in the beverage.

### Proton NMR

The ASEA sample was prepared by adding 550 μL of ASEA and 50 μL of D₂O (Cambridge Isotope Laboratories) to an NMR tube and vortexing the sample for 10 seconds. 1H NMR experiments were performed on a 700 MHz Bruker spectrometer equipped with a QNP cryogenically cooled probe. Experiments used a single pulse with pre-saturation on the water resonance experiment. A total of 1024 scans were taken. All experiments were performed at room temperature.

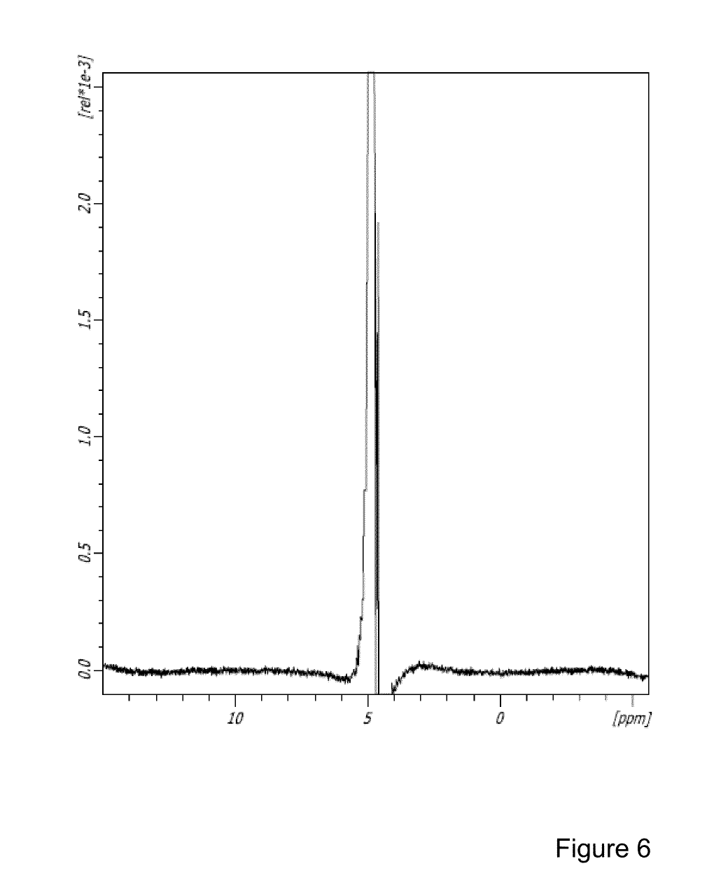

A 1H NMR spectrum of the beverage was collected and is presented in FIG. 6. Only peaks associated with water were able to be distinguished from this spectrum. This spectrum show that very little if any organic material can be detected in the beverage using this method.

### Phosphorous NMR and Mass Spectrometry

DIPPMPO (5-(Diisopropoxyphosphoryl)-5-1-pyrroline-N-oxide) (VWR) samples were prepared by measuring about 5 mg of DIPPMPO into a 2 mL centrifuge tube. This tube then had 550 μL of either the beverage or water added to it, followed by 50 μL of D₂O. A solution was also prepared with the beverage but without DIPPMPO. These solutions were vortexed and transferred to NMR tubes for analysis. Samples for mass spectrometry analysis were prepared by dissolving about 5 mg of DIPPMPO in 600 μL of the beverage and vortexing, then diluting the sample by adding 100 μL of sample and 900 μL of water to a vial and vortexing.

NMR experiments were performed using a 700 MHz Bruker spectrometer equipped with a QNP cryogenically cooled probe. Experiments performed were a single pulse at a P31 frequency of 283.4 MHz. A recycle delay of 2.5 seconds and 16384 scans were used. Phosphoric acid was used as an external standard. All experiments were performed at room temperature.

Mass spectrometry experiments were performed by directly injecting the ASEA/DIPPMPO sample into a Waters/Synapt Time of Flight mass spectrometer. The sample was directly injected into the mass spectrometer, bypassing the LC, and monitored in both positive and negative ion mode.

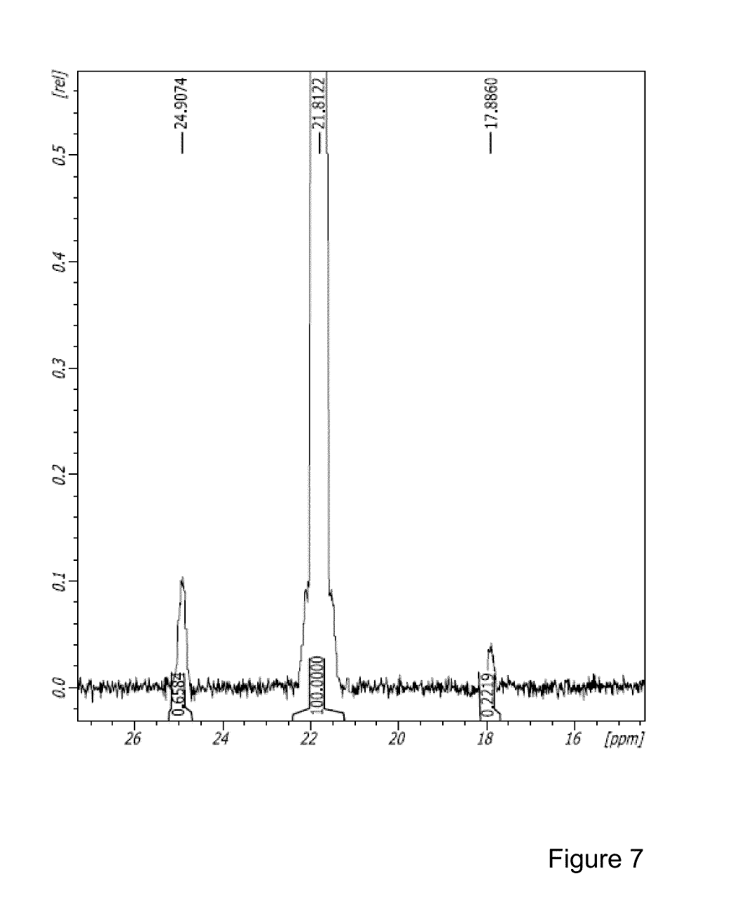

P31 NMR spectra were collected for DIPPMPO in water, the beverage alone, and the beverage with DIPPMPO added to it. An external reference of phosphoric acid was used as a chemical shift reference. FIG. 7 shows the 31P NMR spectrum of DIPPMPO combined with the beverage. The peak at 21.8 ppm was determined to be DIPPMPO and is seen in both the spectrum of DIPPMPO with the beverage (FIG. 7) and without the beverage (not pictured). The peak at 24.9 ppm is most probably DIPPMPO/OH. as determined in other DIPPMPO studies. This peak may be seen in DIPPMPO mixtures both with and without the beverage, but is detected at a much greater concentration in the solution with the beverage. In the DIPPMPO mixture with the beverage, there is another peak at 17.9 ppm. This peak is believed to be from another radical species in the beverage solution. This radical species may be OOH. or possibly a different radical complex. The approximate concentrations of spin trap complexes in the beverage/DIPPMPO solution are as follows:

| Solution | Concentration |
|---|---|
| DIPPMPO | 36.6 mM |
| DIPPMPO/OH• | 241 μM |
| DIPPMPO/radical | 94 μM |

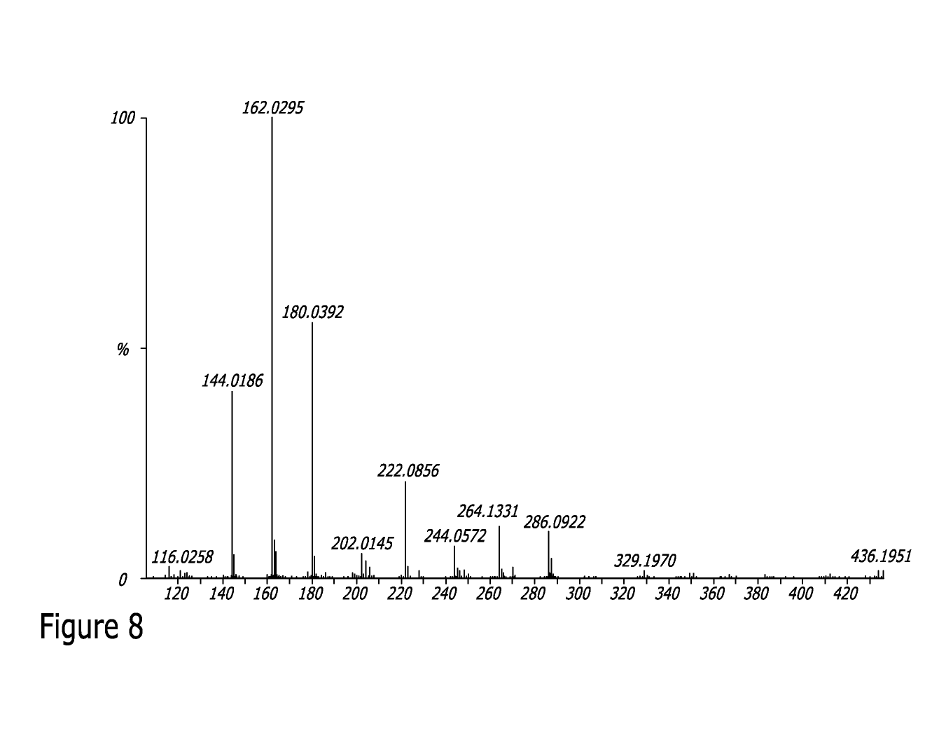

Mass spectral data was collected in an attempt to determine the composition of the unidentified radical species. The mass spectrum shows a parent peak and fragmentation pattern for DIPPMPO with m/z peaks at 264, 222, and 180, as seen in FIG. 8. FIG. 8 also shows peaks for the DIPPMPO/Na adduct and subsequent fragments at 286, 244, and 202 m/z. Finally, FIG. 8 demonstrates peaks for one DIPPMPO/radical complex with m/z of 329. The negative ion mode mass spectrum also had a corresponding peak at m/z of 327. There are additional peaks at 349, 367, and 302 at a lower intensity as presented in FIG. 8. None of these peaks could be positively confirmed. However, there are possible structures that would result in these mass patterns. One possibility for the peak generated at 329 could be a structure formed from a radical combining with DIPPMPO. Possibilities of this radical species include a nitroxyl-peroxide radical (HNO—HOO.) that may have formed in the beverage as a result of reaction with nitrogen from the air. Another peak at 349 could also be a result of a DIPPMPO/radical combination. Here, a possibility for the radical may be hypochlorite-peroxide (HOCl⁻HOO.). However, the small intensity of this peak and small intensity of the corresponding peak of 347 in the negative ion mode mass spectrum indicate this could be a very low concentration impurity and not a compound present in the ASEA solution.

### ICP/MS Analysis

Samples were analyzed on an Agilent 7500 series inductively-coupled plasma mass spectrometer (ICP-MS) in order to confirm the hypochlorite concentration that was determined by NMR. A stock solution of 5% sodium hypochlorite was used to prepare a series of dilutions consisting of 300 ppb, 150 ppb, 75 ppb, 37.5 ppb, 18.75 ppb, 9.375 ppb, 4.6875 ppb, 2.34375 ppb, and 1.171875 ppb in deionized Milli-Q water. These standards were used to establish a standard curve.

Based on NMR hypochlorite concentration data, a series of dilutions was prepared consisting of 164.9835 ppb, 82.49175 ppb, 41.245875 ppb, 20.622937 ppb, 10.311468 ppb, and 5.155734 ppb. These theoretical values were then compared with the values determined by ICP-MS analysis. The instrument parameters were as follows:

| Elements analyzed | 35 Cl, 37 Cl | |
|---|---|---|
| # of points per mass | 20 | |
| # of repetitions | 5 | |
| Total acquisition time | 68.8 | s |
| Uptake speed | 0.50 | rps |
| Uptake time | 33 | s |
| Stabilization time | 40 | s |
| Tune | No Gas | |
| Nebulizer flow rate | 1 | mL/min |
| Torch power | 1500 | W |

The results of the ICP-MS analysis are as follows:

| | Measured Concentration | Concentration by NMR |
|---|---|---|
| Dilution | (ppb) | (ppb) |
| 1 | 81 | 82 |
| 2 | 28 | 41 |
| 3 | 24 | 21 |
| 4 | 13 | 10 |
| 5 | 8 | 5 |

Dilutions were compared graphically to the ICP-MS signals and fit to a linear equation (R²=0.9522). Assuming linear behavior of the ICP-MS signal, the concentration of hypochlorite in the beverage was measured to be 3.02 ppt. Concentration values were determined by calculating the concentration of dilutions of the initial beverage and estimating the initial beverage hypochlorite concentration to be 3 ppt (as determined from 35Cl NMR analysis). The ICP-MS data correlate well with the 35Cl NMR data, confirming a hypochlorite concentration of roughly ⅓% (3 ppt). It should be noted that ICP-MS analysis is capable of measuring total chlorine atom concentration in solution, but not specific chlorine species. The NMR data indicate that chlorine predominantly exists as ClO⁻ in the beverage.

### Gas Phase Quadrupole MS

### Sample Prep

Three sample groups were prepared in triplicate for the analysis: 1) Milli-Q deionized water 2) the beverage, and 3) 5% sodium hypochlorite standard solution. The vials used were 20 mL headspace vials with magnetic crimp caps (GERSTEL). A small stir bar was placed in each vial (VWR) along with 10 mL of sample. The vials were capped, and then placed in a Branson model 5510 sonicator for one hour at 60° C.

The sonicator was set to degas which allowed for any dissolved gasses to be released from the sample into the headspace. After degassing, the samples were placed on a CTC PAL autosampler equipped with a heated agitator and headspace syringe. The agitator was set to 750 rpm and 95° C. and the syringe was set to 75° C. Each vial was placed in the agitator for 20 min prior to injection into the instrument. A headspace volume of 2.5 mL was collected from the vial and injected into the instrument.

### Instrument Parameters

The instrument used was an Agilent 7890A GC system coupled to an Agilent 5975C El/Cl single quadrupole mass selective detector (MSD) set up for electron ionization. The GC oven was set to 40° C. with the front inlet and the transfer lines being set to 150° C. and 155° C. respectively. The carrier gas used was helium and it was set to a pressure of 15 PSI.

The MSD was set to single ion mode (SIM) in order to detect the following analytes:

| Analyte | Mass |
|---|---|
| Water | 18 |
| Nitrogen | 28 |
| Oxygen | 32 |
| Argon | 40 |
| Carbon Dioxide | 44 |
| Chlorine | 70 |
| Ozone | 48 |

The ionization source temperature was set to 230° C. and the quadrupole temperature was set to 150° C. The electron energy was set to 15 V.

Mass spectrometry data was obtained from analysis of the gas phase headspace of the water, the beverage, and hypochlorite solution. The raw area counts obtained from the mass spectrometer were normalized to the area counts of nitrogen in order to eliminate any systematic instrument variation. Both nitrogen and water were used as standards because they were present in equal volumes in the vial with nitrogen occupying the headspace and water being the solvent. It was assumed that the overall volume of water and nitrogen would be the same for each sample after degassing. In order for this assumption to be correct, the ratio of nitrogen to water should be the same for each sample. A cutoff value for the percent relative standard deviation (% RSD) of 5% was used. Across all nine samples, a % RSD of 4.2 was observed. Of note, sample NaClO⁻³ appears to be an outlier, thus, when removed, the % RSD drops to 3.4%.

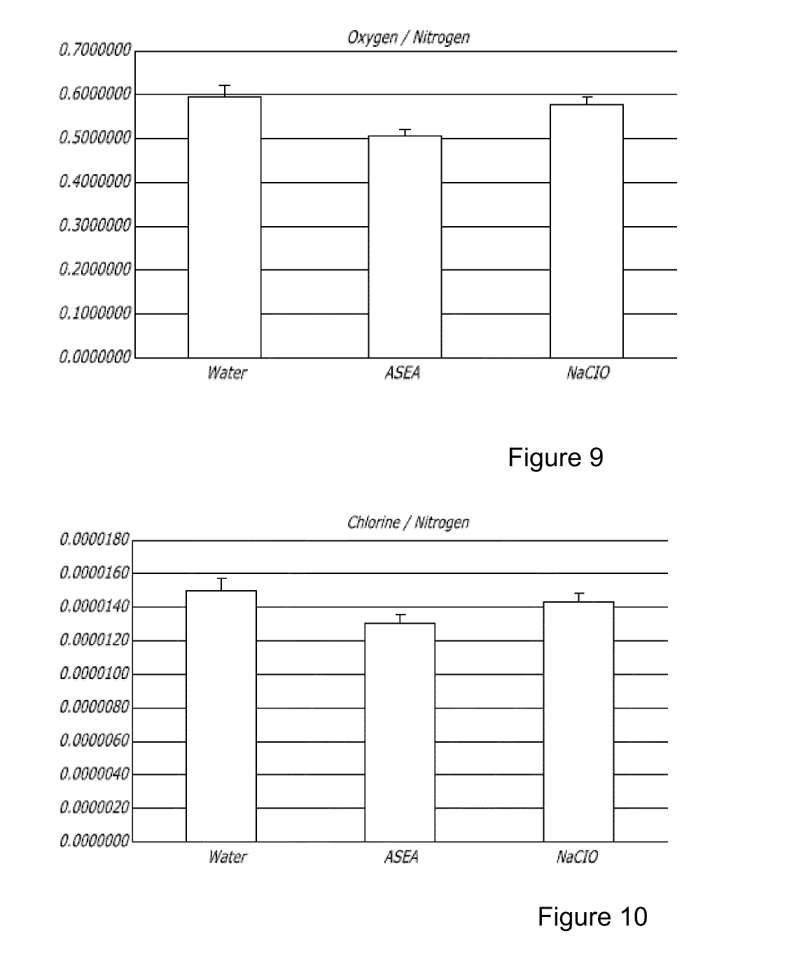

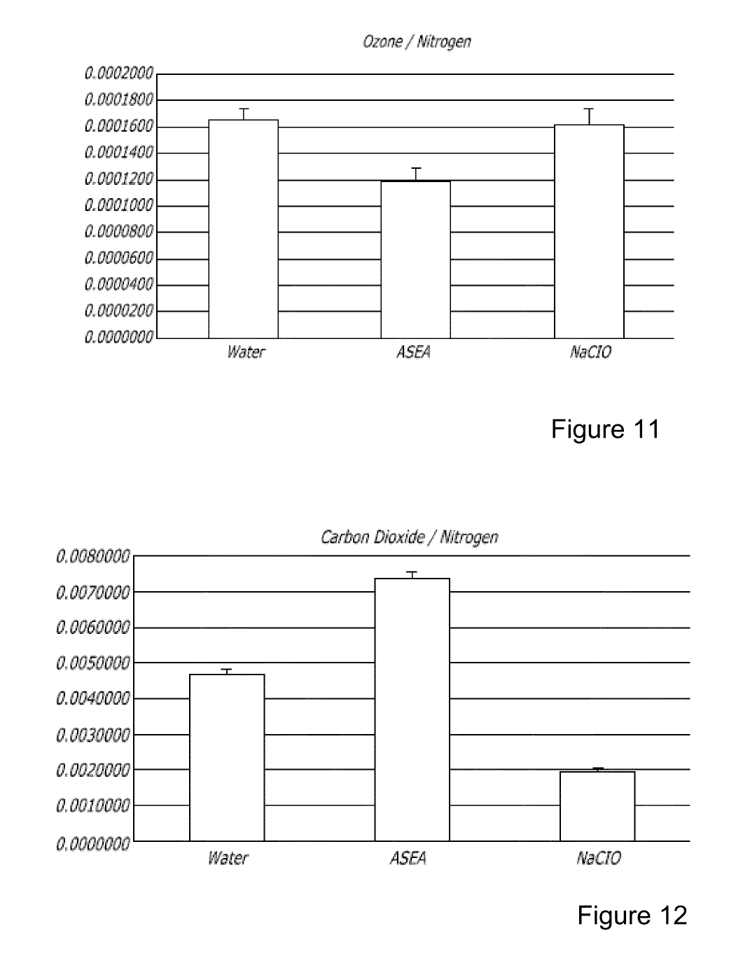

FIGS. 9-11 show the oxygen/nitrogen, chlorine/nitrogen, and ozone/nitrogen ratios. It appears that there were less of these gases released from the beverage than from either water or nitrogen. It should be noted that the signals for both ozone and chlorine were very weak. Thus, there is a possibility that these signals may be due to instrument noise and not from the target analytes.

FIG. 12 shows the carbon dioxide to nitrogen ratio. It appears that there may have been more carbon dioxide released from the beverage than oxygen. However, it is possible that this may be due to background contamination from the atmosphere.

Based on the above, more oxygen was released from both water and sodium hypochlorite than the beverage.

### EPR

Two different beverage samples were prepared for EPR analysis. The beverage with nothing added was one sample. The other sample was prepared by adding 31 mg of DIPPMPO to 20 mL of the beverage (5.9 mM), vortexing, and placing the sample in a 4° C. refrigerator overnight. Both samples were placed in a small capillary tube which was then inserted into a normal 5 mm EPR tube for analysis.

EPR experiments were performed on a Bruker EMX 10/12 EPR spectrometer. EPR experiments were performed at 9.8 GHz with a centerfield position of 3500 Gauss and a sweepwidth of 100 Gauss. A 20 mW energy pulse was used with modulation frequency of 100 kHz and modulation amplitude of 1 G. Experiments used 100 scans. All experiments were performed at room temperature.

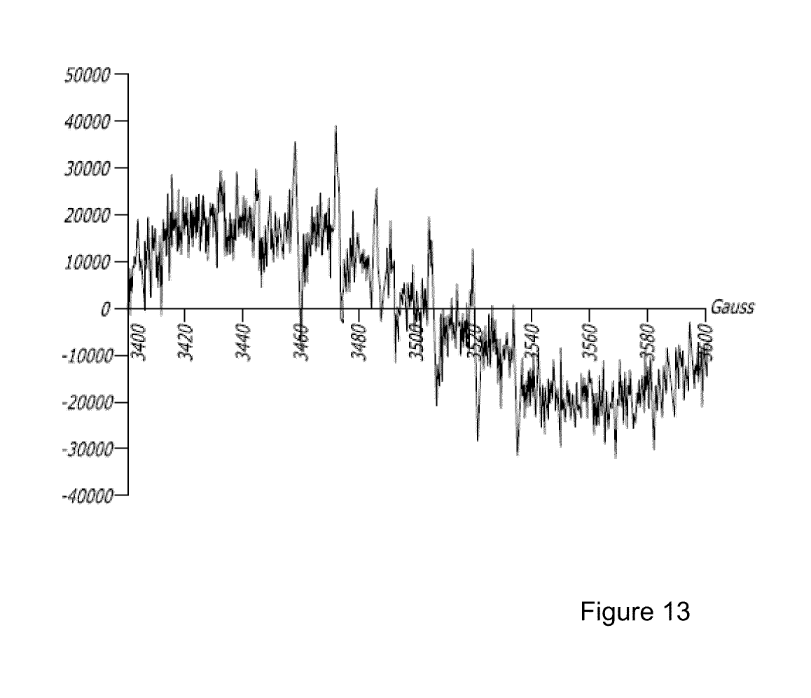

EPR analysis was performed on the beverage with and without DIPPMPO mixed into the solution. FIG. 13 shows the EPR spectrum generated from DIPPMPO mixed with the beverage. The beverage alone showed no EPR signal after 100 scans (not presented). FIG. 13 shows an EPR splitting pattern for a free electron. This electron appears to be split by three different nuclei. The data indicate that this is a characteristic splitting pattern of OH. radical interacting with DMPO (similar to DIPPMPO). This pattern can be described by 14N splitting the peak into three equal peaks and 1H three bonds away splitting that pattern into two equal triplets. If these splittings are the same, it leads to a quartet splitting where the two middle peaks are twice as large as the outer peaks. This pattern may be seen in FIG. 13 twice, with the larger peaks at 3457 and 3471 for one quartet and 3504 and 3518 for the other quartet. In this case, the N14 splitting and the 1H splitting are both roughly 14G, similar to an OH. radical attaching to DMPO. The two quartet patterns in FIG. 13 are created by an additional splitting of 47 G. This splitting is most likely from coupling to 31P, and similar patterns have been seen previously. The EPR spectrum in FIG. 13 indicates that there is a DIPPMPO/OH. radical species in the solution.

### Example 3

### Stability

The oxygen radical content of aqueous formulations was analyzed. In the assay, R-Phycoerythrin [an algal protein] is exposed to varying levels of a standard ROS generating compound [AAPH] wherein the level of fluorescence quenching is logarithmically related to ROS content. This provides a standard curve from which to estimate the ROS content of unknown samples. The levels of ROS in the unknown samples are expressed as mM equivalents of AAPH.

### Materials and Methods

β-PHYCOERYTHRIN OR R-PHYCOERYTHRIN: were purchased from Sigma Chemical Corporation, St. Louis, Mo.

AAPH: 2,2′-azobis(2-amidino-propane) dihydrochloride was purchased from Wako Chemicals USA, Richmond, Va. This compound generates ROS upon contact with water.

FLUORESCENCE READER: an 8 or 16 place fluorescence reader manufactured by Pacific Technologies, Redmond, Wash. was used to detect the fluorescence signal from the phycoerythrins. Temperature was controlled at 37° C. during a 12-20 hr. experimental run. The samples were interrogated every 0.5 to 2 min where each sample interrogation was comprised of 1024 lamp flashes from a LED whose emission spectra was appropriate from the excitation spectra of R-Phycoerythrin. Proper cut-off filters were employed to detect the fluorescence emissions of the phycoerythrins.

### Data Analyses

All data is captured in real time and stored on a laptop computer and upon completion of an experiment, the data is downloaded into an Excel worksheet [Microsoft Corporation, Redmond, Wash.]. The data contained in the worksheet can be manipulated to determine the relative change of fluorescence over the time course of the experiment and subsequently, SigmaPlot Pro v. 7 software [SPSS Software, Chicago, Ill.] is used to determine the area under the curve. The area under the curve [AUC] are plotted against the log 10 mM AAPH concentration to provide a standard curve from which to estimate the levels of ROS in unknown samples.

### Detailed Methods

Step A: To ½″ glass vials, 300 uL of phosphate buffer solution, pH 7.0, 100 mM was added. Then, 15 ug of R-Phycoerythrin in 15 uL of phosphate buffer was added to the phosphate buffer solution. The vials were capped and placed into the wells of the fluorescence reader for 15 min prior to the addition of a saline control, aqueous formulation or AAPH solutions. During this period, fluorescence values were collected from which to calculate a 100% value. This value was then used in subsequent calculations to determine a relative fluorescence signal value for the standard curves.

Step B: Then, 1 mg of AAPH was added to 1 mL of phosphate buffer and 10-fold dilutions were made to provide at least a 3 log 10 range of AAPH concentrations. Similarly, aqueous formulations were diluted and added to appropriate vials in Step B.

Then, 100 uL of the materials in Step A were added to the appropriate vials in Step B. The vials are mixed and replaced into the reader for up to an additional 12 to 20 hrs of evaluation.

### Results/Summary

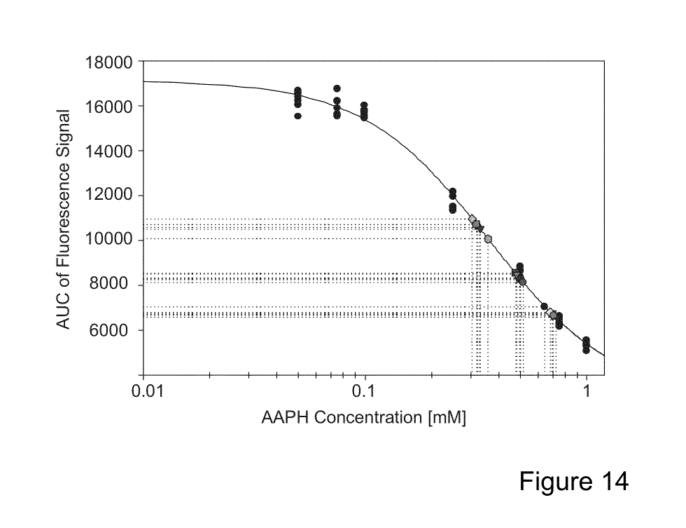

As illustrated in FIG. 14, as the concentration of AAPH decreased from 1.00 mM to 0.050 mM, there was as concomitant increase in the normalized AUC. Buffer control [not shown] revealed that over time there is a spontaneous loss of fluorescence signal, although this loss represents only ˜8% of the original signal.

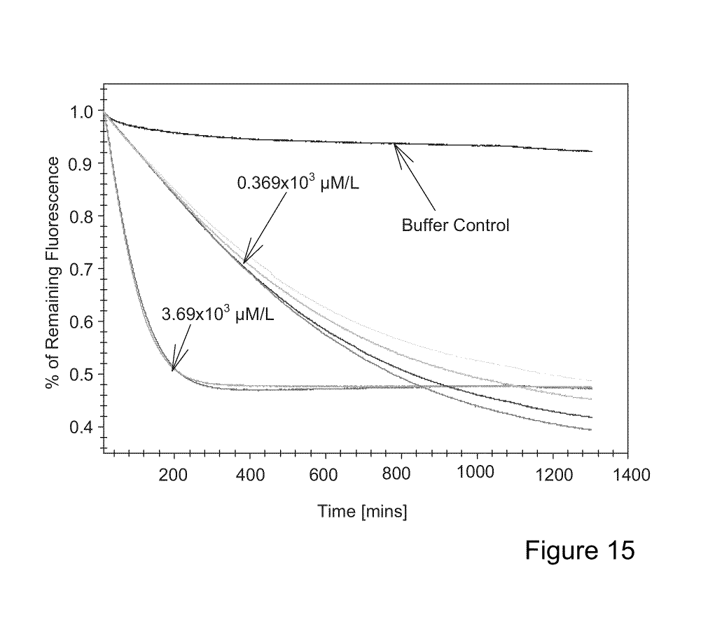

The data represented in FIG. 15 illustrates intra-assay variability of two concentrations of AAPH. Using SigmaStat v2.01 software, the following mean, Std. Deviation and Relative Std. Deviation were calculated and are tabulated below.

| AAPH | AUC Values | | | | |
|---|---|---|---|---|---|
| Concentration | N | Mean | Std. Dev. | Std. Error | % Rel. Std. Dev. |
| 3.69 mM | 3 | 653 | 1.07 | 0.62 | 0.15 |
| 0.369 mM | 4 | 804 | 31.7 | 15.0 | 3.7 |

The data shows that the variation for each concentration among replicates ranged from ˜0.1% to 4% variation [Rel. Std. Dev.]. These data suggest that fluorescence quenching assay is capable of producing small variations among triplicate or quadruplicate samples over a 10-fold range of AAPH concentrations.

The below table shows the results of the analyses of aqueous formulations prepared by MDI and filtered through 0.2 u Supor membrane to ensure sterility prior to clinical application. It is clear that different production lots are similar in their ROS content. Statistical analysis supported this observation [p=0.272]. The most important point is the observation that filtration through a 0.2 u Supor membrane does not decrease the ROS content.

| Treatment | N | Mean AUC | Std. Dev. | Std. Error | % Rel. Std. Dev. |
|---|---|---|---|---|---|
| Unfiltered | 4 | 589.7 | 65.8 | 32.9 | 5.5 |
| Filtered | 4 | 646.3 | 66.3 | 33.1 | 5.1 |

In the below table, data from a typical analysis is illustrated. Saline [negative control] always contained less than 0.1 mM AAPH equivalents of ROS whereas aqueous formulations contained >1.0 mM ROS.

| Aqueous Formulation (AF) | Mean | ROS Content mM |
|---|---|---|
| or Saline Samples | AUC | AAPH equivalents |
| AF | 479 | 3.3 |
| AF | 543 | 2.2 |
| AF | 441 | 4.5 |
| AF | 523 | 2.98 |
| AF | 516 | 3.2 |
| Saline | 974 | 0.095 |
| Saline | 956 | 0.075 |

The development of a phycobiliprotein fluorescence quenching assay for the routine determination of ROS content in aqueous formulations has been successful and is used routinely to monitor production quality for ROS levels. The assay has the following characteristics: ease of use, sensitivity, and quantitation. The assay is linear over a 2 log 10 range of ROS concentrations. For aqueous formulations, the starting saline was used as a negative control, AAPH served as a positive control and allowed the generation of a standard curve, and aqueous formulations or other samples comprised the unknowns.

### Additional Samples Assayed for Stability of the Superoxide Radical

One year long stability studies showed a 3%/month decay rate for superoxide over a 12 month period in polymeric bottles.

Superoxide was analyzed in aqueous formulations in various containers over different time periods. Results are tabulated below.

| | % Potency | % Potency | % Potency |
|---|---|---|---|
| | in Polymer | in Polymer | in Glass |
| Month | Pouch | Bottle | Bottle |
| 0 | 100 | 100 | — |
| 1 | 95 | 94 | — |
| 3 | 92 | 81 | — |
| 8 | — | 56 | — |
| 11 | — | 68 | — |
| 13 | 67 | 60 | — |
| 20 | — | — | 102 |
| 24 | — | — | 93 |

Based on the results above, superoxides were surprisingly found to be more stable in glass bottles after 20 months than in polymeric containers (bottles or pouches) after one month. Even after 24 months, superxoides were 93% stable in glass bottles.

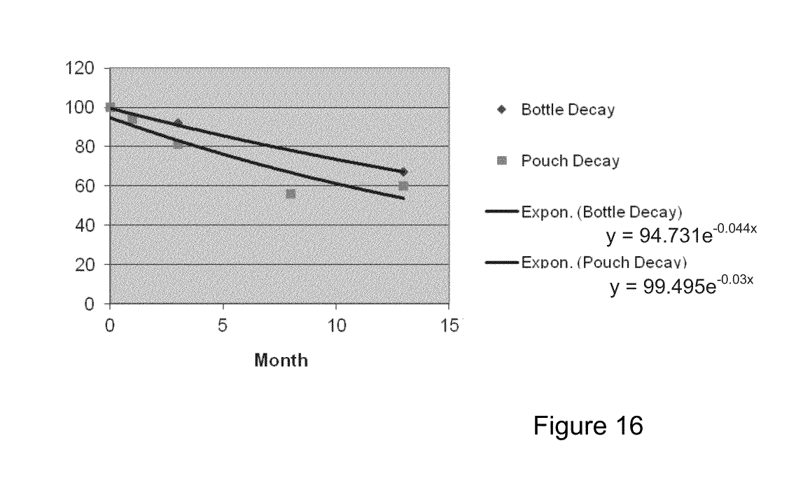

FIG. 16 illustrates decay curves for aqueous formulations stored in plastic bottles versus aqueous formulations stored in polymeric pouches. FIG. 16 shows a 4.4% decay rate of the superoxide radical for the pouch and a 0.3% decay rate for the bottle over a 13 month period. Tests have been performed which show about no decay when using different bottle types. Our tests indicate that bottling the health aqueous formulation in less reactive bottles or bottles that better prevent light from entering and causing decay, allow for a decay rate that approaches and/or is zero.

Both NMR and ESR detect the presence of stable superoxides, hydroxyl radicals and OOH* and confirmatory tests show that the stable superoxides, hydroxyl radicals and OOH* are stable and present in the aqueous formulations.

Unless otherwise indicated, all numbers expressing quantities of ingredients, properties such as molecular weight, reaction conditions, and so forth used in the specification and claims are to be understood as being modified in all instances by the term “about.” Accordingly, unless indicated to the contrary, the numerical parameters set forth in the specification and attached claims are approximations that may vary depending upon the desired properties sought to be obtained by the present invention. At the very least, and not as an attempt to limit the application of the doctrine of equivalents to the scope of the claims, each numerical parameter should at least be construed in light of the number of reported significant digits and by applying ordinary rounding techniques. Notwithstanding that the numerical ranges and parameters setting forth the broad scope of the invention are approximations, the numerical values set forth in the specific examples are reported as precisely as possible. Any numerical value, however, inherently contains certain errors necessarily resulting from the standard deviation found in their respective testing measurements.

The terms “a,” “an,” “the” and similar referents used in the context of describing the invention (especially in the context of the following claims) are to be construed to cover both the singular and the plural, unless otherwise indicated herein or clearly contradicted by context. Recitation of ranges of values herein is merely intended to serve as a shorthand method of referring individually to each separate value falling within the range. Unless otherwise indicated herein, each individual value is incorporated into the specification as if it were individually recited herein. All methods described herein can be performed in any suitable order unless otherwise indicated herein or otherwise clearly contradicted by context. The use of any and all examples, or exemplary language (e.g., “such as”) provided herein is intended merely to better illuminate the invention and does not pose a limitation on the scope of the invention otherwise claimed. No language in the specification should be construed as indicating any non-claimed element essential to the practice of the invention.

Groupings of alternative elements or embodiments of the invention disclosed herein are not to be construed as limitations. Each group member may be referred to and claimed individually or in any combination with other members of the group or other elements found herein. It is anticipated that one or more members of a group may be included in, or deleted from, a group for reasons of convenience and/or patentability. When any such inclusion or deletion occurs, the specification is deemed to contain the group as modified thus fulfilling the written description of all Markush groups used in the appended claims.

Certain embodiments of this invention are described herein, including the best mode known to the inventors for carrying out the invention. Of course, variations on these described embodiments will become apparent to those of ordinary skill in the art upon reading the foregoing description. The inventor expects skilled artisans to employ such variations as appropriate, and the inventors intend for the invention to be practiced otherwise than specifically described herein. Accordingly, this invention includes all modifications and equivalents of the subject matter recited in the claims appended hereto as permitted by applicable law. Moreover, any combination of the above-described elements in all possible variations thereof is encompassed by the invention unless otherwise indicated herein or otherwise clearly contradicted by context.

In closing, it is to be understood that the embodiments of the invention disclosed herein are illustrative of the principles of the present invention. Other modifications that may be employed are within the scope of the invention. Thus, by way of example, but not of limitation, alternative configurations of the present invention may be utilized in accordance with the teachings herein. Accordingly, the present invention is not limited to that precisely as shown and described.

We claim:

1. An aqueous formulation comprising:
an electrolyzed saline solution housed in a non-reactive container, wherein the electrolyzed saline solution includes at least one superoxide radical that is greater than about 60% stable for at least 1 year.

2. The aqueous formulation of claim 1, wherein the non-reactive container is formed of polyethylene, polypropylene, polystyrene, or borosilicate glass.

3. The aqueous formulation of claim 1, wherein the non-reactive container is formed of borosilicate glass.

4. The aqueous formulation of claim 3, wherein the at least one superoxide radical has a half-life of about 24 years.

5. The aqueous formulation of claim 3, wherein the at least one superoxide radical has a half-life of greater than about 24 years.

6. The aqueous formulation of claim 3, wherein the at least one superoxide radical is greater than about 98% stable for at least 1 year.

7. The aqueous formulation of claim 3, wherein the at least one superoxide radical is greater than about 79% stable for at least 6 years.

8. The aqueous formulation of claim 3, wherein the at least one superoxide radical is greater than about 72% stable for at least 8 years.

9. The aqueous formulation of claim 3, wherein the at least one superoxide radical is greater than about 65% stable for at least 10 years.

10. The aqueous formulation of claim 3, wherein the at least one superoxide radical is 100% stable for at least 20 years.

11. The aqueous formulation of claim 1, wherein the electrolyzed saline solution includes the at least one superoxide radical and at least one hypochlorite.

12. The aqueous formulation of claim 1, wherein the electrolyzed saline solution is a product of a circulating solution having a salt concentration of between about 10.00 g NaCl/gal and about 12.00 g NaCl/gal and wherein the circulating solution is electrolyzed using a set of electrodes with an amperage of between about 50 amps and about 60 amps.

13. The aqueous formulation of claim 12, wherein the circulating solution has a temperature of about 4.5° C. to about 5.8° C.

14. A method of making an aqueous formulation comprising:
electrolyzing a circulating solution having a salt concentration of between about 10.00 g NaCl/gal and about 12.00 g NaCl/gal using a set of electrodes with a voltage greater than about 2.6 mV and an amperage of between about 50 amps and about 60 amps thereby forming an electrolyzed saline solution; and
placing the electrolyzed saline solution into a non-reactive container,
wherein the electrolyzed saline solution includes at least one oxygen radical that is greater than about 95% stable for at least 1 year within the non-reactive container.

15. The method of claim 14, wherein the non-reactive container is formed borosilicate glass.

16. The method of claim 14, wherein the oxygen radical is a superoxide.

17. The method of claim 16, wherein the superoxide has a half-life of about 24 years.

18. The method of claim 16, wherein the electrolyzed saline solution includes the superoxide and at least one hypochlorite.

19. The method of claim 14, wherein the at least one oxygen radical is greater than about 98% stable for at least 1 year.

20. The method of claim 14, wherein the at least one oxygen radical is greater than about 79% stable for at least 6 years.

21. The method of claim 14, wherein the at least one oxygen radical is greater than about 72% stable for at least 8 years.

22. The method of claim 14, wherein the at least one oxygen radical is greater than about 65% stable for at least 10 years.

23. The method of claim 14, wherein the at least one oxygen radical is 100% stable for at least 20 years.

24. The method of claim 14, wherein the circulating solution has a temperature of about 4.5° C. to about 5.8° C.

25. The method of claim 14, wherein the aqueous formulation is administered to a mammal to treat a health condition.

26. The method of claim 14, wherein the aqueous formulation is administered to a mammal to enhance an athletic characteristic.
